(19) United States
(12) Patent Application Publication
(10) Pub. No.: US 2010/0311975 A1
(43) Pub. Date: Dec. 9, 2010

(54) CATIONIC TRANSITION METAL CATALYSTS

(76) Inventors: Kamaluddin Abdur-Rashid, Mississauga (CA); Dino Amoroso, Binbrook (CA); Rongwei Guo, Oakville (CA); Xuanhua Chen, Oakville (CA); Christine Sui-Seng, Toronto (CA); Chi-Wing Tsang, Toronto (CA); Wenli Jia, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/739,257

(22) PCT Filed: Oct. 30, 2008
(86) PCT No.: PCT/CA2008/001905

§ 371 Date: Apr. 22, 2010

Related U.S. Application Data
(60) Provisional application No. 60/983,722, filed on Oct. 30, 2007.
(60) Provisional application No. 61/015,434, filed on Dec. 20, 2007.
(60) Provisional application No. 61/049,869, filed on May 2, 2008.

(51) Int. Cl. C07F 15/00; C07F 15/02; C07C 29/143

(52) U.S. Cl. 546/2; 556/13; 556/14; 568/814; 568/812

(57) ABSTRACT

The present disclosure includes catiome complexes of iron, ruthenium, and osmium, and their use as catalysts in organic synthesis transformations including the hydrogenation of unsaturated compounds. The complexes are represented by the following formulae I, II, III, IV, and V, wherein M is Fe, Ru or Os, P is a monodentate ligand with a phosphorus donor atom, P2 is a bidentate neutral ligand with two phosphorus donor atoms, N₂ is a bidentate neutral ligand with two nitrogen donor atoms, PN is a bidentate neutral ligand with phosphorus and nitrogen donor atoms, PNNP is a tetradentate neutral ligand bonded to M via two phosphorus and two nitrogen atoms, X is any anionic ligand, LB is any neutral Lewis base, Y is any non-coordinating anion, n is 0, 1, or 2, m is 1 or 2, q is 0 or 1, r is 1 or 2 and q+r=2.

### FIELD OF THE DISCLOSURE

The present disclosure relates to the field of catalytic reactions, in which a catalytic system comprising a cationic metal complex is used for organic chemical synthesis, for example, but not limited to the hydrogenation or reduction of compounds containing a carbon-carbon (C═C) or a carbon-heteroatom (C═O, C═N) double bond.

### BACKGROUND OF THE DISCLOSURE

The catalysis approach towards synthesis offers several distinct advantages (e.g. cost savings, less waste generation) over more traditional protocols using stoichiometric reagents. In particular, transition metal (TM) catalysis has revolutionized organic synthesis (Tsuji, J. Transition Metal Reagens and Catalysts; Wiley: West Sussex, England, 2002). The near constant improvement in the field of TM catalysis is undoubtedly due in large part to the introduction of new and improved ligands, which allows for desired transformations to be carried out in a more efficient manner (i.e. milder conditions, lower catalyst loadings, higher yields and higher enantioselectivities when applicable).

Catalytic hydrogenation is one of the fundamental reactions in chemistry, and is used in a large number of chemical processes. It is now recognized that catalytic hydrogenations of carbon-carbon double bonds of alkenes, and carbon-heteroatom double bonds of ketones, aldehydes and imines are indispensable processes for the production of the wide range of alkanes, alcohols and amines, including chiral compounds, which are useful as valuable end products and precursors for the pharmaceutical, agrochemical, flavor, fragrance, material and fine chemical industries.

Amongst the several different kinds of processes known to achieve such transformation, two important types are: (a) transfer hydrogenation processes, in which hydrogen-donors such as secondary alcohols, and in particular isopropanol (ⁱPrOH), and triethlammonium formate (HCOOH/NEt₃) are used, (b) hydrogenation processes, in which molecular hydrogen is used. Both hydrogen transfer and hydrogenation processes need a catalyst or catalytic system to activate the reducing agent, such as an alcohol, HCOOH/NEt₃ or molecular hydrogen.

The catalytic hydrogenation processes developed by Noyori and coworkers (Ohkuma et al., J. Am. Chem. Soc., 1995, 107, 2675 and 10417) are very attractive, since the catalysts consist of air-stable ruthenium complexes of the type RuCl₂(PR₃)₂(diamine) and RuCl₂(diphosphine)(diamine) which are precursors for the generation of what appears to be some of the most active catalysts for the homogeneous and asymmetric hydrogenation of ketones and imines in the presence of a base and hydrogen gas. It has been proposed and subsequently mechanistically elucidated that the key molecular recognition feature of these catalysts is the presence of mutually cis N—H and Ru—H moieties of the catalytic dihydride species (RuH₂(PR₃)₂(diamine) and RuH₂(diphosphine)(diamine)) that electronically bind and activate the substrate and facilitate reduction.

Other reactions for which transition metal catalysts have found significant applications include hydroformylations, hydrosilylations, hydroborations, hydroaminations, hydrovinylations, hydroarylations, hydrations, oxidations, epoxidations, reductions, C—C and C—X bond formations (includes reactions such as Heck, Suzuki-Miyaura, Negishi, Buchwald-Hartwig Amination, α-Ketone Arylation, N-Aryl Amination, Murahashi, Kumada, Negishi and Stille reactions etc.), functional group interconversions, kinetic resolutions, dynamic kinetic resolutions, cycloadditions, Diels-Alder reactions, retro-Diels-Alder reactions, sigmatropic rearrangements, electrocyclic reactions, ring-opening and/or ring-closing olefin metatheses, carbonylations, and aziridinations.

### SUMMARY OF THE DISCLOSURE

The hydrogenation of ketones has been successfully and advantageously performed using cationic salts of certain neutral Fe(II), Ru(II) and Os(II) complexes. The cationic complexes were prepared by treatment of the neutral precursors with anion abstracting agents. The resulting complexes are air and moisture stable. Solutions can be prepared and handled in air with no obvious signs of decay. The activity of the cationic complexes matches that of the neutral precursors. In several cases, the cationic derivatives give products with improved enantiomeric excess relative to the neutral congener.

Accordingly, the present disclosure provides a compound selected from a compound of Formula I, II, III, IV and V:

[M(P₂)(PN)X_{q}(LB)ₙ]^{r+}[Y⁻]ᵣ (I)

[M(PN)₂X_{q}(LB)ₙ]^{r+}[Y⁻]ᵣ (II)

[M(P)ₘ(N₂)X_{q}(LB)ₙ]^{r+}[Y⁻]ᵣ (III)

[M(PNNP)X_{q}(LB)ₙ]^{r+}[Y⁻]ᵣ (IV) and

[M(P₂)(N₂)X_{q}(LB)ₙ]^{r+}[Y⁻]ᵣ (V)

wherein
M is Fe, Ru or Os;
P is a monodentate ligand bonded to M via a phosphorus atom;
P₂ is a bidentate neutral ligand bonded to M via two phosphorus atoms;
N₂ is a bidentate neutral ligand bonded to M via two nitrogen atoms;
PN is a bidentate neutral ligand bonded to M via a phosphorus atom and a nitrogen atom;
PNNP is a tetradentate neutral ligand bonded to M via two phosphorus and two nitrogen atoms;
X is any anionic ligand;
LB is any neutral Lewis base;
Y is any non-coordinating anion;
n is 0, 1 or 2;
m is 1 or 2; and
q is 0 or 1;
r is 1 or 2; and
q+r=2.

Also included in the present disclosure is a process for preparing a compound of the disclosure comprising combining a precursor metal compound, an anion abstracting agent, and one or more P, P₂, N₂, PN or PNNP ligands, and optionally a base and reacting under conditions to form the compound of the disclosure and optionally isolating the compound of the disclosure.

The present disclosure also includes a method for catalyzing a synthetic organic reaction comprising combining starting materials for the reaction with a compound according to the disclosure under conditions for performing the reaction.

The present disclosure also includes the use of a compound of the disclosure for catalyzing a synthetic organic reaction.

The synthetic organic transformations to which the compounds of the disclosure can be applied include but are not limited to: hydrogenations, transfer hydrogenations, hydroformylations, hydrosilylations, hydroborations, hydroaminations, hydrovinylations, hydroarylations, hydrations, oxidations, epoxidations, reductions, C—C and C—X bond formations (including, for example, Heck, Suzuki-Miyaura, Negishi, Buchwald-Hartwig Amination, α-Ketone Arylation, N-Aryl Amination, Murahashi, Kumada, Negishi and Stille reactions etc.), functional group interconversions, kinetic resolutions, dynamic kinetic resolutions, cycloadditions, Diels-Alder reactions, retro-Diels-Alder reactions, sigmatropic rearrangements, electrocyclic reactions, ring-openings, ring-closings, olefin metatheses, carbonylations, and aziridinations. In all transformations listed above the reactions may or may not be regioselective, chemoselective, stereoselective or diastereoselective.

In an embodiment, the present disclosure relates to a process for the reduction of compounds comprising a carbon-carbon (C═C), carbon-oxygen (C═O) or carbon-nitrogen (C═N) double bond, to the corresponding hydrogenated alkane, alcohol or amine, comprising contacting a compound comprising the C═C, C═O or C═N double bond with a catalyst of the Formula (I), (II), (III), (IV) or (V) under hydrogenation conditions.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the attached drawings in which:

FIG. 1 is an X-ray crystal structure of [RuCl(pyridine)(R-binap)(R,R-cydn)]BF₄. Hydrogen atoms, BF₄ anion and two CHCl₃ molecules omitted for clarity.

### DETAILED DESCRIPTION OF THE DISCLOSURE

### (I) Definitions

The term “C₁₋ₙalkyl” as used herein means straight and/or branched chain, saturated alkyl radicals containing from one to “n” carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the largest number of carbon atoms in the alkyl radical.

The term “C₁₋ₙalkenyl” as used herein means straight and/or branched chain, unsaturated alkyl radicals containing from one to n carbon atoms and one to three double bonds, and includes (depending on the identity of n) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable n is an integer representing the largest number of carbon atoms in the alkenyl radical.

The term “C₃₋ₙcycloalkyl” as used herein means a monocyclic or polycyclic saturated carbocylic group containing from three to n carbon atoms and includes (depending on the identity of n), cyclopropyl, cyclobutyl, cyclopentyl, cyclodecyl, bicyclo[2.2.2]octane, bicyclo[3.1.1]heptane and the like, where the variable n is an integer representing the largest number of carbon atoms in the cycloalkyl group.

The term “aryl” as used herein means a monocyclic, bicyclic or tricyclic aromatic ring system containing at least one aromatic ring and from 6 to 14 carbon atoms and includes phenyl, naphthyl, anthracenyl, 1,2-dihydronaphthyl, 1,2,3,4-tetrahydronaphthyl, fluorenyl, indanyl, indenyl and the like.

The term “heterocyclic” as used herein means a monocyclic, bicyclic or tricyclic ring system containing from 5 to 14 atoms of which, unless otherwise specified, one, two, three, four or five are heteromoieties independently selected from N, NR^{a}, NR^{b}R^{c}, O, S, SiR^{a} and SiR^{b}R^{c}, wherein R^{a} is selected from H, C₁₋₆alkyl, ═O and OH and R^{b} and R^{c} are independently selected from H and C₁₋₆alkyl. When the ring system includes at least one aromatic ring it is referred to as “heteroaryl”. Heterocylic groups include, for example, thienyl, furyl, pyrrolyl, pyrididyl, indolyl, quinolyl, isoquinolyl, tetrahydroquinolyl, benzofuryl, benzothienyl and the like.

The term “halo” as used herein means halogen and includes chloro, fluoro, bromo, iodo and the like.

The term “fluoro-substituted” as used herein means that one or all of the hydrogens on the referenced group is replaced with fluorine.

The suffix “ene” added on to any of the above groups means that the group is divalent, i.e. inserted between two other groups.

The term “ring system” as used herein refers to a carbon-containing ring system, that includes monocycles, fused bicyclic and polycyclic rings, bridged rings and metalocenes. Where specified, the carbons in the rings may be substituted or replaced with heteroatoms.

The term “polycyclic” as used herein means groups that contain more than one ring linked together and includes, for example, groups that contain two (bicyclic), three (tricyclic) or four (quadracyclic) rings. The rings may be linked through a single bond, a single atom (spirocyclic) or through two atoms (fused and bridged).

The term “non-coordinating anion” as used herein refers to an anion which does not formally bond to or share electrons with the metal center in a covalent bond.

The term “joined together” as used herein means that two substituents are linked together via a linker grouping to form a ring system. The linker grouping comprises at least one atom but may also comprise several atoms, for example up to 20 atoms, resulting in the formation of monocyclic and polycyclic ring systems.

The term “compound(s) of the disclosure” means a compound of the Formula (I), (II), (III), (IV) or (V), or mixtures thereof.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

Rendering the neutral metal complexes of the present disclosure into an ionic pair dramatically altered the behaviour and properties of the original metal complex. These changes may be borne out of changes in structure of the resulting complex, the charged nature of the newly formed ionic complex or they may be a result of qualities imparted by the new counter ion. Regardless of the origin of effect, there were great advantages gained from this approach in the present disclosure.

Removal of any coordinating ligand from the metal complexes of the present disclosure had the effect of introducing a vacant coordination site. In transition-metal catalysis this is often imperative for substrate binding and may indeed be rate limiting with respect to the catalytic cycle. Abstraction of one or two anionic ligands and substituting them with non- or weakly coordinating anions represents one such method for installing a vacant coordination site. In this manner, generating cationic complexes by abstraction of coordinating anionic ligands and substitution with non-coordinating anionic ligands lead to more active catalysts.

The exchange of one or two coordinating anionic ligands with non-coordinating or weakly coordinating ligands resulted in a more electrophilic, cationic metal centre. This increased electrophilicity lead to stronger binding between the metal and nucleophilic substrates. With respect to catalytic processes involving metal-substrate interactions, this has the obvious consequences and is especially beneficial in the case of weaker nucleophiles such as those with electron-withdrawing groups.

Transforming the covalent metal complexes of the present disclosure into ionic salts lead to derivatives which were more stable than their parents. Without wishing to be limited by theory, increased stability is the result of the removal of electron density from the metal leading to a metal centre which is less readily oxidized. Thus, the ionic salts prepared from neutral precursors were generally more stable to oxidation under atmospheric conditions displaying greater tolerance toward oxygen and moisture and greater storage stability (i.e. shelf-life).

The solubility properties of ionic complexes were also different from their neutral precursors. Generally, ionic complexes tended to be more soluble in polar solvents and less soluble in apolar solvents. Some ionic complexes were also more soluble in aqueous solutions. That being said, the solubility of the ionic complex can be further tuned with the selection of the anion. For instance, highly fluorinated anions tended to impart a high degree of solubility in a broad range of solvents. In fact, many ionic complexes incorporating highly fluorinated anions were more soluble in nonpolar solvents than the corresponding neutral precursor while their solubility in polar solvents remained high owing to the ionic nature of the complex.

The ability to tailor solubility also afforded the ability to control the solid properties of the ionic complex. That is, polar salts could be readily precipitated with nonpolar solvents leading to higher isolated yields and more regular and controllable particle sizes. A corollary to this property is that these ionic catalysts also hold the promise of more facile removal from product mixtures. An obvious benefit when one considers the use of ionic catalysts in applications where low residual metals are imperative.

While rendering a neutral catalyst cationic holds the promise of many critical advantages, the utility of this approach is limited by competence in catalysis of the resulting ionic complex. If the derived ionic catalyst is no longer active in catalysis then the advantages described above are obviously moot. In the present disclosure, the cationic ruthenium catalysts were shown to be excellent hydrogenation catalysts. The activity of the cationic complexes matched that of the neutral precursors and, in several cases, the cationic derivatives gave products with improved enantiomeric excess relative to the neutral congener. While not wishing to be limited by theory, this is likely due to the fact that the cationic complexes disclosed herein are more reliably and reproducibly activated prior to entering the catalytic cycle. That is to say that while all of the complexes are subject to activation, the cationic complexes fare better in this process than the neutral analogues. The activation process, which is carried out in alcohol solvents and is often irreproducible and unpredictable, is better suited to the cationic complexes since they are soluble in the solvent system while the neutral complexes are either insoluble or moderately soluble. The poor solubility of the neutral compounds means that the activation is often incomplete and can lead to side reactions giving catalytically inactive species or active species which do not retain the desired stereoselectivity.

Accordingly, the present disclosure provides a compound selected from a compound of Formula I, II, III, IV and V:

[M(P₂)(PN)X_{q}(LB)ₙ]^{r+}[Y⁻]ᵣ (I)

[M(PN)₂X_{q}(LB)ₙ]^{r+}[Y⁻]ᵣ (II)

[M(P)ₘ(N₂)X_{q}(LB)ₙ]^{r+}[Y⁻]ᵣ (III)

[M(PNNP)X_{q}(LB)ₙ]^{r+}[Y⁻]ᵣ (IV) and

[M(P₂)(N₂)X_{q}(LB)ₙ]^{r+[Y}⁻]ᵣ (V)

wherein
M is Fe, Ru or Os;
P is a monodentate ligand bonded to M via a phosphorus atom;
P₂ is a bidentate neutral ligand bonded to M via two phosphorus atoms;
N₂ is a bidentate neutral ligand bonded to M via two nitrogen atoms;
PN is a bidentate neutral ligand bonded to M via a phosphorus atom and a nitrogen atom;
PNNP is a tetradentate neutral ligand bonded to M via two phosphorus and two nitrogen atoms;
X is any anionic ligand;
LB is any neutral Lewis base;
Y is any non-coordinating anion;
n is 0, 1 or 2;
m is 1 or 2;
q is 0 or 1;
r is 1 or 2; and
q+r=2.

In an embodiment of the disclosure, P is a monodentate phosphine ligand of the Formula (VI):

PR¹R²R³ (VI)

wherein R¹, R² and R³ are independently selected from C₆₋₁₈aryl, C₁₋₂₀alkyl and C₃₋₂₀cycloalkyl, each being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl. In further embodiments of the disclosure, R¹, R² and R³ are independently selected from phenyl, C₁₋₆alkyl and C₃₋₁₀cycloalkyl, each being optionally substituted with one to three substituents independently selected from C₁₋₄alkyl, fluoro-substituted C₁₋₄alkyl, halo, C₁₋₄alkoxy and fluoro-substituted C₁₋₆alkoxy. In further embodiments of the disclosure, R¹, R² and R³ are all cyclohexyl, phenyl, xylyl or tolyl.

In another embodiment of the disclosure, P₂ is a bidentate bisphosphino ligand of the Formula (VII):

R⁴R⁵P-Q¹-PR⁶R⁷ (VII)

wherein R⁴, R⁵, R⁶ and R⁷ are, independently, as defined for R¹, R² and R³, and Q¹ is selected from unsubstituted or substituted C₁-C₁₀alkylene and unsubstituted or substituted C₁-C₈alkenylene where the substituents on Q¹ are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl and/or two substituents on Q¹ are joined together to form, including the carbon atoms to which they are attached, one or more unsubstituted or substituted 5-20-membered monocyclic, polycyclic, heterocyclic, carbocyclic, saturated, unsaturated or metallocenyl ring systems, and Q¹ is chiral or achiral. In further embodiments of the disclosure, R⁴, R⁵, R⁶ and R⁷ are independently selected from phenyl, C₁₋₆alkyl and C₃₋₁₀cycloalkyl, each being optionally substituted with one to three substituents independently selected from C₁₋₄alkyl, fluoro-substituted C₁₋₄alkyl, halo, C₁₋₄alkoxy and fluoro-substituted C₁₋₄alkoxy and Q¹ is selected from unsubstituted or substituted C₁-C₈alkylene where the substituents on Q¹ are independently selected from one to four C₁₋₄alkyl, fluoro-substituted C₁₋₄alkyl halo, C₁₋₄alkoxy, fluoro-substituted C₁₋₄alkoxy, unsubstituted and substituted phenyl and substituted and unsubstituted naphthyl, or two substituents are joined together to form, including the carbon atoms to which they are attached, one or more unsubstituted or substituted phenylene, cyclohexylene, naphthylene, pyridylene or ferrocenylene groups, and Q¹ is chiral or achiral. In further embodiments of the disclosure, R⁴, R⁵, R⁶ and R⁷ are all cyclohexyl, phenyl, xylyl or tolyl. Unless otherwise specified, the term substituted means that one or more, including all, but suitably one to five, of the available hydrogen atoms on a group are replaced with C₁₋₆alkyl, fluoro-substituted C₁₋₆alkyl, C₁₋₆alkoxy, fluoro-substituted C₁₋₄alkyl, halo or C₆₋₁₄aryl. Representative examples of the preparation of bis(phosphino) ligands are found in Gupta, M. et al. Chem. Commun. 1996, 2083-2084; Moulton, C. J. J. Chem. Soc. Dalton, 1976, 1020-1024). Other bis(phosphino) ligands are selected from:
2,2′-bis-(diphenylphosphino)-1,1′-binaphthyl (BINAP);
2,2′-bis(diphenylphosphino)-5,5′,6,6′,7,7′,8,8′-octahydro-1,1′-binaphthyl (H₈BINAP);
2,2′-bis-(diphenylphosphino)-6,6′-dimethyl-1,1′-binaphthyl (6MeBINAP);
2,2′-bis-(di-p-tolylphosphino)-1-,1′-binaphthyl (Tol-BINAP);
2,2′-bis[bis(3-methylphenyl)phosphino]-1,1′-binaphthyl;
2,2′-bis[bis(3,5-di-tert-butylphenyl)phosphino]-1,1′-binaphthyl;
2,2′-bis[bis(4-tert-butylphenyl)phosphino]-1,1′-binaphthyl;
2,2′-bis[bis(3,5-dimethylphenyl)phosphino]-1,1′-binaphthyl (Xyl-BINAP);
2,2′-bis[bis(3,5-dimethyl-4-methoxyphenyl)phosphino]-1,1′-binaphthyl (Dmanyl-BINAP);
2,2′-bis[bis-(3,5-dimethylphenyl)phosphino]-6,6′-dimethyl-1,1′-binaphthyl (Xyl-6MeBINAP);
3,3′-bis-(diphenylphosphanyl)-13,13′-dimethyl-12,13,14,15,16,17,12′,13′,14′,15′,16′,17′-dodecahydro-11H,11′H-[4,4′]bi[cyclopenta[a]phenanthrenyl];

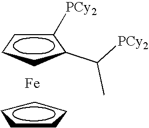

wherein Cy is C₅₋₈cycloalkyl;

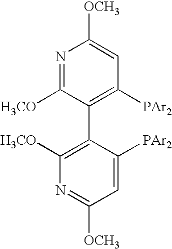

where Ar is phenyl (PPhos), xylyl (XylPPhos) or tolyl (TolPPhos);

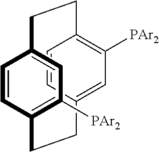

where Ar is phenyl (PhanePhos), xylyl (XylPhanePhos) or tolyl (TolPhanePhos); and optical isomers thereof and mixtures of optical isomers in any ratio.

In another embodiment of the disclosure, PN is a ligand of the Formula (VIII):

R⁸R⁹P-Q²-NR¹⁰R¹¹ (VIII)

wherein R⁸ and R⁹ are, independently as defined for R¹-R³;
Q² is as defined for Q¹; and
R¹⁰ and R¹¹ are independently selected from H, C₆₋₁₈aryl, C₁₋₂₀alkyl and C₃₋₁₀cycloalkyl, each being optionally substituted with one to five substituents independently selected from C₁₋₆alkyl, fluoro-substituted C₁₋₆alkyl halo, C₁₋₆alkoxy, fluoro-substituted C₁₋₆alkoxy and C₆₋₁₄aryl, or
R¹⁰ and R¹¹ are joined to form, together with the nitrogen atom to which they are attached, a saturated, unsaturated or aromatic, monocyclic or polycyclic, substituted or unsubstituted ring system containing from 3 to 14 atoms, or
one of R¹⁰ or R¹¹ are joined with a substituent on Q² to form, together with the nitrogen atom to which R¹⁰ and R¹¹ is attached, a 4- to 10-membered saturated, unsaturated or aromatic, monocyclic or bicyclic ring system, where if the nitrogen atom is part of aromatic ring or is bonded to an adjacent atom via a double bond, the other of R¹⁰ and R¹¹ is non-existent. In embodiments of the disclosure, R⁸ and R⁹ are independently selected from phenyl, C₁₋₆alkyl and fluoro-substituted C₁₋₆alkyl, with the phenyl being optionally substituted with one to five substituents independently selected from C₁₋₄alkyl, fluoro-substituted C₁₋₄alkyl halo, C₁₋₄alkoxy and fluoro-substituted C₁₋₄alkoxy and Q² is selected from unsubstituted or substituted C₁-C₈alkenylene where the substituents on Q² are independently selected from one to four of C₁₋₆alkyl, fluoro-substituted C₁₋₆alkyl, halo, C₁₋₆alkoxy, fluoro-substituted C₁₋₆alkoxy and unsubstituted or substituted phenyl and/or two substituents on Q² are joined together to form, including the carbon atoms to which they are attached, one or more unsubstituted or substituted phenylene, naphthylene or ferrocenylene ring systems, and Q² is chiral or achiral. In further embodiments of the disclosure, R⁸ and R⁹ are all phenyl, tolyl or xylyl. In further embodiments, R¹⁰ and R¹¹ and both H. In a further embodiment, one of R¹⁰ or R¹¹ is joined with a substituent on Q² to form, together with the nitrogen atom to which R¹⁰ and R¹¹ is attached, a substituted or unsubstituted pyridine ring and the other of one of R¹⁰ or R¹¹ is not present. Unless otherwise specified, the term substituted means that one or more, including all, but suitably one to five, of the available hydrogen atoms on a group are replaced with C₁₋₆alkyl, fluoro-substituted C₁₋₆alkoxy, fluoro-substituted C₁₋₆alkoxy, halo or C₆₋₁₄aryl. Examples of PN ligands, include, for example, Ph₂PCH₂CH₂NH₂ (abbreviated as PGly), and:

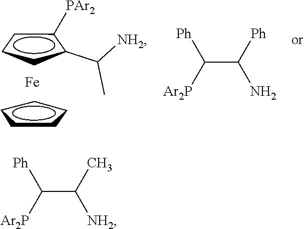

wherein Ar is selected from Ph, tolyl and xylyl, and optical isomers thereof and mixtures of optical isomers.

In a further embodiment of the disclosure, PNNP is a tetradentate diaminodiphosphine of the formula (IXa) or a diiminodiphosphine ligand of the Formula (IXb):

R¹²R¹³P-Q³-NR¹⁴-Q⁴-NR¹⁵-Q⁵-PR¹⁶R¹⁷ (IXa)

R¹²R¹³P-Q³=N-Q⁴-N=Q⁵-PR¹⁶R¹⁷ (IXb)

wherein R¹², R¹³, R¹⁶ and R¹⁷ are independently as defined for R¹-R³, R¹⁴ and R¹⁵ are independently as defined for R¹⁰ and R¹¹ and Q³, Q⁴ and Q⁵ are independently as defined for Q¹. In further embodiments of the disclosure, R¹², R¹³, R¹⁶ and R¹⁷ are independently selected from phenyl, C₁₋₆alkyl and C₃₋₁₀cycloalkyl, each being optionally substituted with one to five substituents independently selected from C₁₋₄alkyl, fluoro-substituted C₁₋₄alkyl, halo, C₁₋₄alkoxy and fluoro-substituted C₁₋₆alkoxy and Q³, Q⁴ and Q⁵ are independently selected from unsubstituted or substituted C₁-C₈alkylene and from unsubstituted or substituted C₁-C₈alkenylene, where the substituents on Q³, Q⁴ and Q⁵ are independently selected from one to four C₁₋₄alkyl, fluoro-substituted C₁₋₄alkyl, halo, C₁₋₆alkoxy, fluoro-substituted C₁₋₆alkoxy, unsubstituted and substituted phenyl and substituted and unsubstituted naphthyl or two substituents are joined together to form, including the carbon atoms to which they are attached, one or more unsubstituted or substituted phenyl, cyclohexyl, naphthyl or ferrocenyl groups, and Q³, Q⁴ and Q⁵ are chiral or achiral. In further embodiments of the disclosure, R¹², R¹³, R¹⁶ and R¹⁷ are all phenyl, tolyl or xylyl. Unless otherwise specified, the term substituted means that one or more, including all, but suitably one to five, of the available hydrogen atoms on a group are replaced with C₁₋₆alkyl, fluoro-substituted C₁₋₆alkyl, C₁₋₆alkoxy, fluoro-substituted C₁₋₆alkoxy, halo or C₆₋₁₄aryl. Representative examples of the preparation of diaminodiphosphine ligands are found in Li, Y-Y. et al. 2004, 218, 153-156. Exemplary PNNP ligands include:

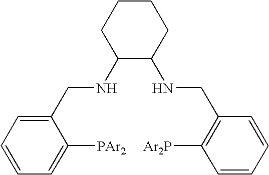

wherein Ar is phenyl (abbreviated as DPPcydn), tolyl (abbreviated as di(p-tolyl)PPcydn) or xylyl (abbreviated as di(3,5xylyl)PPcydn);

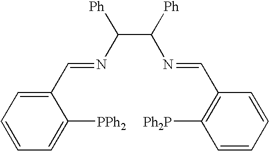

abbreviated as dpenPPh₂N₂, and each optical isomer thereof and mixtures of optical isomers.

In another embodiment of the disclosure, N₂ is a bidentate diamine ligand of the Formula (X):

R¹⁸R¹⁹N-Q⁶-NR²⁰R²¹ (X)

wherein R¹⁸, R¹⁹, R²⁰ and R²¹ are independently as defined for R¹⁰ and R¹¹ and Q⁶ is as defined for Q¹, or one of R¹⁸ or R¹⁹ and/or R²⁰ or R²¹ are joined with a substituent on Q⁶ to form, together with the nitrogen atom to which R¹⁸, R¹⁹, R²⁰ or R²¹ is attached, a 4- to 10-membered saturated, unsaturated or aromatic, monocyclic or bicyclic, substituted or unsubstituted ring system, where if the nitrogen atom is part of aromatic ring or is bonded to an adjacent atom via a double bond, the other of R¹⁸ or R¹⁹ and/or R²⁰ or R²¹ is non-existent. In embodiments of the disclosure, R¹⁸, R¹⁹, R²⁰ and R²¹ are all H and Q⁶ is selected from unsubstituted or substituted C₁-C₈alkenylene where the substituents on Q⁶ are independently selected from one to four of C₁₋₆alkyl, fluoro-substituted C₁₋₆alkyl, halo, C₁₋₆alkoxy, fluoro-substituted C₁₋₆alkoxy and unsubstituted or substituted phenyl and/or two substituents on Q⁶ are joined together to form, including the carbon atoms to which they are attached, one or more unsubstituted or substituted phenyl, naphthyl or ferrocenyl ring systems, and Q⁶ is chiral or achiral. In a further embodiment, one of R¹⁸ or R¹⁹ or R²⁰ or R²¹ are joined with a substituent on Q⁶ to form, together with the nitrogen atom to which R¹⁸, R¹⁹, R²⁰ or R²¹ is attached, a substituted or unsubstituted pyridine ring and the other of one of R¹⁸ or R¹⁹ and/or R²⁰ or R²¹ is not present. Unless otherwise specified, the term substituted means that one or more, including all, but suitably one to five, of the available hydrogen atoms on a group are replaced with C₁₋₆alkyl, fluoro-substituted C₁₋₆alkyl, C₁₋₆alkoxy, fluoro-substituted C₁₋₆alkoxy, halo or C₆₋₁₄aryl. Examples of the diamine ligands include, for example, methylenediamine, ethylenediamine, 1,2-diaminopropane, 1,3-diaminopropane, 1,4-diaminobutane, 2,3-diaminobutane, 1,2-cyclopentanediamine, 1,2-cyclohexanediamine, 1,1-diphenylethylenediamine, 1,1-di(p-methoxyphenyl)ethylenediamine, 1,1-di(3,5-dimethoxyphenyl)ethylenediamine, and 1,1-dinaphthylethylenediamine. Optically active diamine compounds may be also used. Examples thereof include, for example, each optical isomer of 1,2-diphenylethylenediamine (abbreviated name: DPEN), 1,2-di(p-methoxyphenyl)ethylenediamine, 1,2-cyclohexanediamine, 1,2-cycloheptanediamine, 2,3-dimethylbutanediamine, 1-methyl-2,2-diphenylethylenediamine (abbreviated as DACH or CYDN), 1-isobutyl-2,2-diphenylethylenediamine, 1-isopropyl-2,2-diphenylethylenediamine, 1-benzyl-2,2-diphenylethylen-ediamine, 1-methyl-2,2-di(p-methoxyphenyl)ethylenediamine (abbreviated name: DAMEN), 1-isobutyl-2,2-di(p-methoxyphenyl)-ethylenediamine (abbreviated name: DAIBEN), 1-isopropyl-2,2-di(p-methoxyphenyl)ethylenediamine (abbreviated name: DAIPEN), 1-benzyl-2,2-di(p-methoxyphenyl)ethylenediamine, 1-methyl-2,2-di(3,5-dimethoxyphenyl)ethylenediamine, 1-isopropyl-2,2-di(3,5-dimethoxyphenyl)ethylenediamine, 1-isobutyl-2,2-di(3,5-dimethoxy-phenyl)ethylenediamine, 1-benzyl-2,2-di(3,5-dimethoxyphenyl)ethylenediamine, 1-methyl-2,2-dinaphthylethylenediamine, 1-isobutyl-2,2-dinaphthylethylene- diamine, 1-isopropyl-2,2-dinaphthylethylenediamine, and 1-benzyl-2,2-dinaphthylethylenediamine, and mixtures of optical isomers in any ratio. Further, optically active diamine compounds which can be used are not limited to the abovementioned optically active ethylenediamine derivatives. Optically active propanediamine, butanediamine and cyclohexanediamine derivatives may be also used. In addition, these diamine ligands may be prepared by the process starting from a-amino acids described in the literature (Burrows, C. J., et al., Tetrahedron Letters, 34(12), pp. 1905-1908 (1993)), or by a variety of processes described in the general remark (T. Le Gall, C. Mioskowski, and D. Lucet, Angew. Chem. Int. Ed., 37, pp. 2580-2627 (1998)). In another embodiment of the disclosure, N₂ is the bidentate aminopyridine ligand:

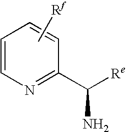

wherein R^{e} is H, C₁₋₆alkyl, fluoro-substituted C₁₋₆alkyl or C₆₋₁₄aryl, R^{f} is H, halo, C₁₋₆alkyl, fluoro-substituted-C₁₋₆alkyl, C₂₋₆alkenyl, C₂₋₆alkynyl, C₃₋₇cycloalkyl, C₁₋₆alkoxy, fluoro-substituted-C₁₋₆alkoxy or C₆₋₁₄aryl, and including each optical isomer thereof and mixtures of optical isomers. In another embodiment, R^{f} is H, halo, C₁₋₄alkyl, fluoro-substituted-C₁₋₄alkyl, C₂₋₄alkenyl, C₂₋₄alkynyl, C₃₋₇cycloalkyl, C₁₋₄alkoxy, fluoro-substituted-C₁₋₄alkoxy or phenyl.

In an embodiment of the disclosure, X is any suitable anionic ligand, including, but not limited to, halo, C₁₋₆alkoxy, carboxylate, sulfonates and nitrates. Suitably X is Cl.

LB is any suitable neutral Lewis base, for example any neutral two electron donor, for example acetonitrile, DMF, pyridine, tetrahydrofuran (THF), CO, tBuCN or t-BuNC.

Y is any non-coordinating counter anion, including, but not limited to, OTf, BF₄, PF₆, B(C₁₋₆alkyl)₄, B(fluoro-substituted-C₁₋₆alkyl)₄ or B(C₆₋₁₈aryl)₄ wherein C₆₋₁₈aryl is unsubstituted or substituted 1-5 times with fluoro, C₁₋₄alkyl or fluoro-substituted C₁₋₄alkyl. In another embodiment, Y is

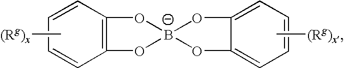

wherein R^{g} is independently halo, C₁₋₄alkyl, fluoro-substituted-C₁₋₄alkyl or C₆₋₁₈aryl and x and x′ are independently an integer between 1 and 4. In another embodiment, R^{g} is halo, suitably fluoro. In a further embodiment, Y is

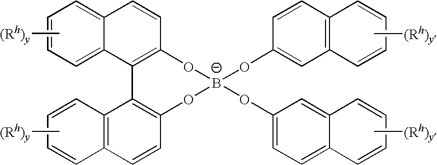

wherein R^{h} is independently halo, C₁₋₄alkyl, fluoro-substituted-C₁₋₄alkyl or C₆₋₁₈aryl and y and y′ are independently an integer between 1 and 6. In another embodiment, R^{h} is halo, suitably fluoro. In another embodiment, Y is Al(C₁₋₆alkyl)₄, Al(fluoro-substituted-C₁₋₆alkyl)₄, Al(C₆₋₁₈aryl)₄, Al(—O—C₁₋₆alkyl)₄, Al(—O-fluoro-substituted-C₁₋₆alkyl)₄ or Al(—O—C₆₋₁₈aryl)₄, wherein C₆₋₁₈aryl is unsubstituted or substituted 1-5 times with halo, C₁₋₄alkyl or fluoro-substituted C₁₋₄alkyl. In a further embodiment, Y is a carborane or a bromocarborane anion. In another embodiment, the carborane anion is a carborane such as CB₁₁H₁₂. In another embodiment, the bromocarborane is a bromocarborane such as CB₁₁H₆Br₆.

In a further embodiment, Y is a phosphate anion. In a further embodiment the phosphate anion is of the formula

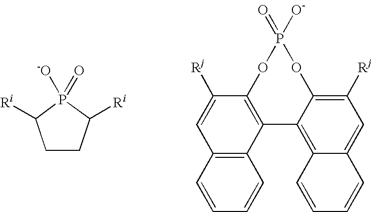

wherein Rⁱ and R^{j} are independently selected from halo, C₁₋₄alkyl, fluoro-substituted-C₁₋₄alkyl or C₆₋₁₈aryl.

In an embodiment, the anion Y is a chiral compound and is optically pure.

In general, one or two anionic ligands bound to the neutral metal precursor is abstracted by treatment with a salt of a non-coordinating anion (i.e. one which does not formally bond to or share electrons with the metal centre in a typical covalent bond) suitably in an inert atmosphere at ambient or room temperature. This leads to the formation of a salt complex comprised of a formally cationic metal complex and the associated, non- or weakly coordinating anion(s). Exemplified below (Scheme 1, reaction 1) is the use of dichloride ruthenium precursor complexes however this methodology is easily extended to other, non-chloride and other metal-containing precursors. Indeed, any other halide precursor can be handled analogously while similar procedures can be employed for non-halide precursors such as carboxylates, sulfonates, nitrates etc. Exposure of the resulting cationic complexes to coordinating Lewis Bases, either during the anion abstraction/metathesis reaction or by treatment of the isolated salts, leads to the formation of a coordinatively-saturated metal adduct. In an embodiment, after formation of the cationic catalysts, adducts are formed by the addition of co-ordinating Lewis Bases. This is described in general terms below in reaction 2. The corresponding dicationic complexes (i.e. where both anionic ligands are removed) function in a similar manner.

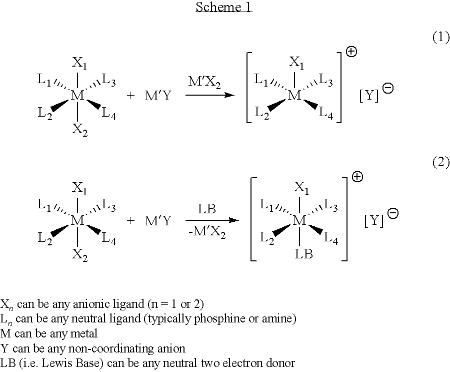

In another embodiment, the formation of the compounds of the disclosure is via a procedure wherein a precursor to the neutral complexes, is first rendered cationic or dicationic by treatment with one or two equivalents of a salt of a non-coordinating anion and then treated with the appropriate ligand to generate the compounds of the disclosure. Also, a one-pot procedure can also be envisioned where all of the components are combined to generate the cationic transition-metal complexes.

Accordingly, the present disclosure further includes a process for preparing a compound of the disclosure comprising combining a compound of the formula

M(P₂)(PN)X₂ (XI)

M(PN)₂X₂ (XII)

M(P)ₘ(N₂)X₂ (XIII)

M(PNNP)X₂ (XIV) or

M(P₂)(N₂)X₂ (XV)

wherein M, P₂, PN, P, PNNP, P₂ and X are as defined above, with one or two molar equivalents of an anion abstracting agent and optionally a non- or weakly-coordinating Lewis Base, and reacting under conditions to form the compound of the disclosure and optionally isolating the compound of the disclosure.

In a further embodiment of the present disclosure, there is included a process for preparing a compound of the disclosure comprising combining a precursor metal compound with one or two molar equivalents of an anion abstracting agent, and optionally a Lewis Base and reacting under conditions to form a cationic or dicationic precursor metal compound and combining the cationic or dicationic precursor metal compound with one or more P, P₂, N₂, PN, or PNNP ligands, as defined above, under conditions to form the compound of the disclosure and optionally isolating the compound of the disclosure.

In an embodiment of the disclosure, the precursor metal compound is of the formula [MX₂(p-ligand)]₂ or MX₂(ligand) wherein M and X are as defined for the compounds of the disclosure and ligand is any displaceable ligand, for example, p-cymene, benzene, cyclooctadiene (COD) or norbornadiene (NBD), suitably p-cymene or norbornadiene (NBD), for example [MCl₂(p-cymene)]₂ or [MCl₂(NBD)]ₙ wherein M is a metal selected from Fe, Ru and Os, in particular ruthenium.

In another embodiment of the disclosure, the precursor metal compound is of the formula MX₂(P₂)(LB)ₙ, wherein M, X, P₂ and LB are as defined above and n is 1 or 2. In an embodiment, the precursor metal compound is readily converted into its cationic counterparts [MX(P₂)(LB)ₙ]Y or [M(P₂)(LB)ₙ]Y₂, by treatment with one or two molar equivalents of an anion abstracting agent as defined above. The corresponding cation is an air stable solid which is isolated in high yields and stored under ambient conditions. A cationic compound of the formula [MX(P₂)(LB)ₙ]Y or [M(P₂)(LB)ₙ]Y₂ is readily converted into the cationic catalysts of the present disclosure, for example, by reaction with one or more P₂, N₂ or PN ligands, as defined above. In an embodiment, (P₂) is BINAP and LB is DMF or pyridine. Metal-diphosphine-DMF complexes have been reported in the literature (Noyori et al. Tetrahedron Lett. 1991, 32:4163).

In another embodiment, the anion abstracting agent is a salt of a non-coordinating counter anion Y as defined above. In yet another embodiment, the ligands are selected from one or more of a compound of the Formula (VI), (VII), (VIII), (IX) and (X) as defined above. In another embodiment, the conditions to form the compound of the disclosure comprise reacting at a temperature of about 20° C. to about 200° C., suitably about 50° C. to about 100° C. in a suitable solvent, for about 30 minutes to 48 hours, following by cooling to room temperature. In an embodiment of the disclosure, the compound of the disclosure is isolated using standard techniques, such as by filtration, evaporation of the solvent, recrystallization and/or chromatography, to provide the compound of Formula (I), (II), (III), (IV) or (V).

### (III) Processes Utilizing the Compounds of the Disclosure

The compounds of the present disclosure are useful as catalysts in organic synthesis transformations. Accordingly, the present disclosure also includes a method for catalyzing a synthetic organic reaction comprising combining starting materials for the reaction with a compound according to the disclosure under conditions for performing the reaction.

The present disclosure also includes the use of a compound of the disclosure for catalyzing a synthetic organic reaction.

In an embodiment of the disclosure, the synthetic organic reaction is selected from hydrogenation, transfer hydrogenation, hydroformylation, hydrosilylation, hydroboration, hydroamination, hydrovinylation, hydroarylation, hydration, oxidation, epoxidation, reduction, C—C and C—X bond formation (including for example, Heck, Suzuki-Miyaura, Negishi, Buchwald-Hartwig Amination, α-Ketone Arylation, N-Aryl Amination, Murahashi, Kumada, Negishi and Stille reactions), functional group interconversion, kinetic resolution, dynamic kinetic resolution, cycloaddition, Diels-Alder, retro-Diels-Alder, sigmatropic rearrangement, electrocyclic reactiona, ring-opening and/or ring-closing olefin metathesis, carbonylation and aziridination. The reaction conditions for these synthetic transformation are well known to those skilled in the art.

In one particular embodiment of the present disclosure, the compounds of the present disclosure are competent hydrogenation (including transfer hydrogenation) catalysts (as can be seen from the tables of experimental data included herein). The complexes are air and moisture stable. Solutions can be prepared and handled in air with no obvious signs of decay. The activity of the cationic complexes matches that of the neutral precursors. In several cases, the cationic derivatives give products with improved enantiomeric excess relative to the neutral congener (compare entry 27 to 28 and 29 and entry 30 to 31 and 32 in Table 1). While not wishing to be limited by theory, this is likely due to the fact that the cationic complexes disclosed herein are more reliably and reproducibly activated prior to entering the catalytic cycle. That is to say that while all of the complexes are subject to activation, the cationic complexes fare better in this process than the neutral analogues. The activation process, which is carried out in alcohol solvents and is often irreproducible and unpredictable, is better suited to the cationic complexes since they are soluble in the solvent system while the neutral complexes less so. The poor solubility of the neutral compounds means that the activation is often incomplete and can lead to side reactions giving catalytically inactive species or active species which do not retain the desired stereoselectivity.

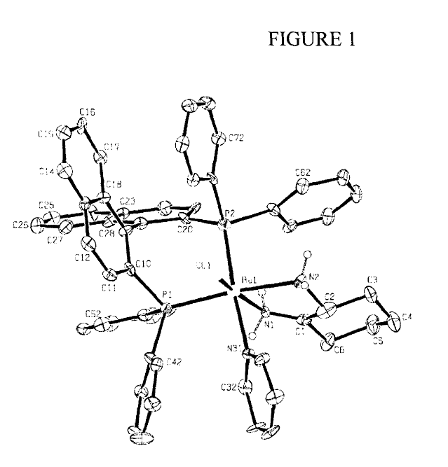

An interesting result to come out of the derivatization to charged species is in the ligand rearrangement observed in the solid state structure of [RuCl(pyridine)(R-binap)(R,R-cydn)]BF₄ (vide infra). The X-ray crystal structure of this compound shows that one of the P atoms of the BINAP ligand is trans to the coordinated pyridine ligand (FIG. 1). (It is believed that this is occurs even in the absence of a Lewis base). This is in contrast to the precursor, RuCl₂(R-binap)(R,R-cydn), where both P atoms are trans to the N atoms of the diamine ligand. Such ligand rearrangements are believed to be important processes during activation of the catalysts and may account for superior activity and selectivity of the cationic catalysts relative to their neutral analogues.

Accordingly, the present disclosure relates to a process for the reduction of compounds comprising a carbon-carbon (C═C), carbon-oxygen (C═O) or carbon-nitrogen (C═N) double bond, to the corresponding hydrogenated alkane, alcohol or amine, comprising contacting a compound comprising the C═C, C═O or C═N double bond with a catalyst of the Formula (I), (II), (III), (IV) or (V) under hydrogenation conditions.

The compound comprising a C═C, C═O or C═N, includes compounds having one or more C═C, C═O and/or C═N bonds.

In an embodiment of the invention, the compound comprising a carbon-oxygen (C═O) or carbon-nitrogen (C═N) double bond is a compound of Formula (XI):

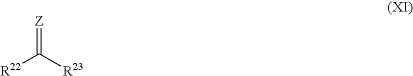

wherein,
Z is selected from CR²⁴R²⁵, NR²⁶, (NR²⁶R²⁷)⁺D⁻ and O;
R²² and R²³ are simultaneously or independently selected from H, aryl, C₁₋₂₀alkyl, C₂₋₂₀alkenyl, C₃₋₂₀cycloalkyl and heteroaryl, said latter 5 groups being optionally substituted;
R²⁴ to R²⁷ are independently or simultaneously selected from H, OH, C₁₋₂₀alkoxy, aryloxy, C₁₋₂₀alkyl, C₂₋₂₀alkenyl, C₃₋₂₀cycloalkyl and aryl, said latter 6 groups being optionally substituted;
or
one or more of R²² to R²⁷ are linked to form, together with the atoms to which they are attached, an optionally substituted ring system; and
D⁻ represents a counteranion,
   wherein heteroaryl is a mono- or bicyclic heteroaromatic group containing from 5 to 10 atoms, of which 1-3 atoms is a heteroatom selected from the group consisting of S, O and N, and wherein the optional substituents are selected from the group consisting of halo, OH, NH₂, OR²⁸, NR²⁸₂ or R²⁸ groups, in which R²⁸ is selected from C₁₋₆alkyl, C₂₋₆alkenyl and aryl and one or more of the carbon atoms in the alkyl, alkenyl and cycloalkyl groups may be optionally replaced with a heteromoiety selected from O, S, N, NH and NC₁₋₄alkyl.

Reduction of compounds of Formula (XI) using a compound of the disclosure according to the process described above provides the corresponding hydrogenated compounds of Formula (XII):

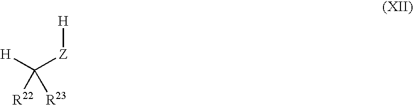

wherein Z, R²² and R²³ are defined as in Formula (XII).

Since R²² and R²³ may be different, it is hereby understood that the final product of Formula (XII), may be chiral, thus possibly consisting of a practically pure enantiomer or of a mixture of stereoisomers, depending on the nature of the catalyst used in the process.

In an embodiment of the disclosure, the hydrogenation conditions characterizing the above process may comprise a base. Said base can be the substrate itself, if the latter is basic, or any conventional base. One can cite, as non-limiting examples, organic non-coordinating bases such as DBU, an alkaline or alkaline-earth metal carbonate, a carboxylate salt such as sodium or potassium acetate, or an alcoholate or hydroxide salt. In an embodiment of the disclosure, the bases are the alcoholate or hydroxide salts selected from the group consisting of the compounds of formula (R³⁰O)₂M′ and R³⁰OM″, wherein M′ is an alkaline-earth metal, M″ is an alkaline metal and R³⁰ stands for hydrogen or a linear or branched C₁₋₂₀alkyl group.

Standard hydrogenation conditions, as used herein, typically implies the mixture of the substrate with a metal complex of Formula (I), (II), (III), (IV) or (V) with or without a base, possibly in the presence of a solvent, and then treating such a mixture with a hydrogen donor solvent at a chosen pressure and temperature (transfer hydrogenation) or in an atmosphere of hydrogen gas at a chosen pressure and temperature. Varying the reaction conditions, including for example, temperature, pressure, solvent and reagent ratios, to optimize the yield of the desired product would be well within the abilities of a person skilled in the art.

The following non-limiting examples are illustrative of the present disclosure:

### Examples

The disclosure will now be described in further details by way of the following examples, wherein the temperatures are indicated in degrees centigrade and the abbreviations have the usual meaning in the art. All the procedures described hereafter have been carried out under an inert atmosphere unless stated otherwise. All preparations and manipulations were carried out under H₂, N₂ or Ar atmospheres with the use of standard Schlenk, vacuum line and glove box techniques in dry, oxygen-free solvents. Tetrahydrofuran (THF), diethyl ether (Et₂O), methylene chloride and hexanes were obtained using an IT solvent purification system. Deuterated solvents were degassed and dried over activated molecular sieves. NMR spectra were recorded on a 300 MHz spectrometer (300 MHz for ¹H, 75 MHz for ¹³C and 121.5 for ³¹P). All ³¹P chemical shifts were measured relative to 85% H₃PO₄ as an external reference. ¹H and ¹³C chemical shifts were measured relative to partially deuterated solvent peaks but are reported relative to tetramethylsilane.

### Example 1

### Synthesis of Cationic Ruthenium Precursors

### (a) [RuCl(p-cymene)]₂[BF₄]₂

In an Ar filled flask, 0.25 g (0.041 mmol) [RuCl₂(p-cymene)]₂ and 0.16 g (0.082 mmol) of AgBF₄ were combined. CH₂Cl₂ (10 mL) was added and the resulting orange suspension was left to stir at ambient temperature. Within several minutes the suspension darkened to brown/green in colour. After 2 hours, the suspension was filtered through Celite and the orange filtrate was reduced to approximately 1 mL in volume. Addition of hexane afforded an oily orange solid which was washed repeatedly with hexane and dried in vacuo. Yield: 0.215 g (74%).

### Example 2

### Synthesis of Cationic Ruthenium Hydrogenation Catalysts

### (a) [RuCl(R-binap)(R,R-cydn)]BF₄

In an Ar filled flask, 0.600 g (0.66 mmol) of RuCl₂(R-binap)(R,R-cydn) and 0.129 g (0.66 mmol) of AgBF₄ were combined. CH₂Cl₂ (15 mL) was added and the resulting rust coloured suspension was left to stir at ambient temperature for two hours after which time it was filtered, in air, through Celite. The orange filtrate was reduced to dryness leaving an orange residue. Yield: 0.620 g (97%). ³¹P NMR (ppm, CDCl₃): 7.53 (d, J_{PP}=45 Hz), 67.5 (d, J_{PP}=45 Hz).

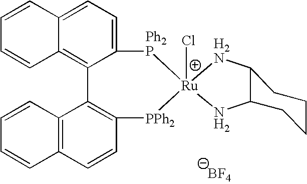

### (b) [RuCl(R-binap)(Ph₂PCH₂CH₂NH₂)]BF₄

In an Ar filled flask, 0.766 g (0.73 mmol) of RuCl₂(R-binap)(Ph₂PCH₂CH₂NH₂) and 0.143 g (0.73 mmol) of AgBF₄ were combined. CH₂Cl₂ (15 mL) was added and the resulting dark orange suspension was left to stir at ambient temperature for two hours after which time it was filtered, in air, through Celite. The dark orange filtrate was reduced to dryness leaving a deep orange residue. Yield: 0.790 g (98%). ³¹P NMR (ppm, CDCl₃): 32.6 (dd, J_{PP}=31 Hz, J_{PP}=24 Hz), 48.0 (dd, J_{PP}=34 Hz, J_{PP}=31 Hz), 62.7 (dd, J_{PP}=34 Hz, J_{PP}=24 Hz).

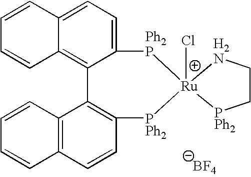

### (c) [RuCl(Ph₂PCH₂CH₂NH₂)₂]BF₄

In an Ar filled flask, 0.750 g (1.19 mmol) of RuCl₂(Ph₂PCH₂CH₂NH₂)₂ and 0.232 g (1.19 mmol) of AgBF₄ were combined. CH₂Cl₂ (15 mL) was added and the resulting red suspension was left to stir at ambient temperature for two hours after which time it was filtered, in air, through Celite. The dark red filtrate was reduced to dryness leaving a deep red residue. Yield: 0.790 g (97%). ³¹P NMR (ppm, acetone-D₆): 55.0 (d, J_{PP}=36 Hz), 73.3 (d, J_{PP}=36 Hz).

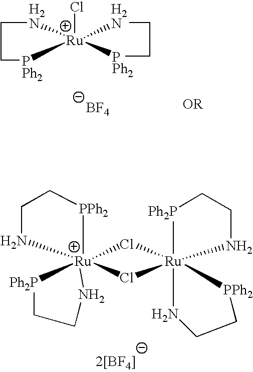

### (d) [RuCl(MeCN)(R-binap)(R,R-cydn)]BF₄

In an Ar filled flask, 0.150 g (0.16 mmol) of [RuCl(R-binap)(R,R-cydn)]BF₄ was dissolved in 6 mL of CH₂Cl₂ and 41 mL (0.78 mmol) of MeCN was added and the brown solution was left to stir. After 16 hours the solution had changed to pale green in colour. Removal of an aliquot for subsequent ³¹P NMR analysis showed that no starting material remained. Concentration of the solvent to approximately 1 mL followed by the addition of hexane (10 mL) afforded a pale green solid. The solid was filtered off, washed with hexane (2×5 mL) and dried in vacuo. Yield: 0.127 g (81%). NMR analysis of the isolated solid revealed the presence of several isomeric species, the major constituent accounting for 80% of the integrated intensity. NMR data are given only for the major isomer. ³¹P NMR (ppm, CDCl₃): 46.3 (d, J_{PP}=34 Hz), 48.8 (d, J_{PP}=34 Hz).

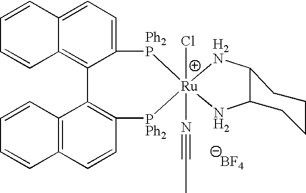

### (e) [RuCl(pyridine)(R-binap)(R,R-cydn)]BF₄

In an Ar filled flask, 0.150 g (0.16 mmol) of [RuCl(R-binap)(R,R-cydn)]BF₄ was dissolved in 6 mL of CH₂Cl₂ and 63 mL (0.78 mmol) of pyridine was added and the brown solution was left to stir. After 16 hours the solution had changed to yellow in colour. Removal of an aliquot for subsequent ³¹P NMR analysis showed that no starting material remained. Concentration of the solvent to approximately 1 mL followed by the addition of hexane (10 mL) afforded a yellow solid. The solid was filtered off, washed with hexane (2×5 mL) and dried in vacuo Yield: 0.152 g (94%). NMR analysis of the isolated solid revealed two complexes; one identified as the desired product (NMR data given below) and the other as starting material (see above). The two compounds were present in approximately equal amounts. It is unclear if a single product is isolated and dissociation of bound pyridine occurs upon dissolution or if reversion to starting material occurs during isolation. ³¹P NMR (ppm, CDCl₃): 55.2 (d, J_{PP}=37 Hz), 49.4 (d, J_{PP}=37 Hz).

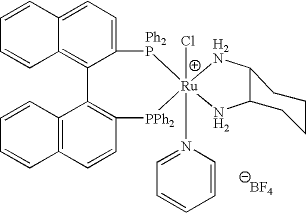

### (f) [RuCl(R-bina_{P})(S,S-Ph₂PCH(Ph)CH(Ph)NH₂]BF₄

In an Ar filled flask, 0.150 g (0.13 mmol) of RuCl₂(R-binap)(S,S-Ph₂PCH(Ph)CH(Ph)NH₂) and 0.025 g (0.13 mmol) of AgBF₄ were combined. CH₂Cl₂ (6 mL) was added and the resulting green suspension was left to stir at ambient temperature for sixteen hours over which time it changed to brown in colour. The suspension was filtered, in air, through Celite and the brown filtrate was reduced to dryness leaving a brown residue. Yield: 0.108 g (69%). ³¹P NMR (ppm, CD₂Cl₂): 34.5 (pseudo t, J_{PP}=27 Hz), 44.5 (dd, J_{PP}=31 Hz, J_{PP}=28 Hz), 83.9 (dd, J_{PP}=31 Hz, J_{PP}=26 Hz).

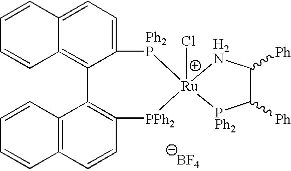

### (g) [RuCl(S-binap)(S,S-Ph₂PCH(PNCH(Ph)NH₂]BF₄

In an Ar filled flask, 0.150 g (0.13 mmol) of RuCl₂(S-binap)(S,S-Ph₂PCH(Ph)CH(Ph)NH₂) and 0.025 g (0.13 mmol) of AgBF₄ were combined. CH₂Cl₂ (6 mL) was added and the resulting green suspension was left to stir at ambient temperature for sixteen hours over which time it changed to brown in colour. The suspension was filtered, in air, through Celite and the brown filtrate was reduced to dryness leaving a brown residue. Yield: 0.096 g (61%). ³¹P NMR (ppm, CD₂Cl₂): 26.7 (dd, J_{PP}=27 Hz, J_{PP}=20 Hz), 48.5 (dd, J_{PP}=33 Hz, J_{PP}=27 Hz), 86.3 (dd, J_{PP}=33 Hz, J_{PP}=20 Hz).

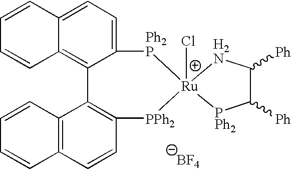

### (h) [RuCl(R-binap)(R,R-cydn)][B(C₆F₅)₄]

In an Ar filled flask, 0.200 g (0.22 mmol) of RuCl₂(R-binap)(R,R-cydn) and 0.192 g (0.22 mmol) of [Li(OEt₂)_{2.5}][B(C₆F₅)₄] were combined. CH₂Cl₂ (10 mL) was added and the resulting dark orange suspension was left to stir at ambient temperature for 16 hours after which time it was filtered, in air, through Celite. The orange filtrate was reduced to dryness leaving an orange residue. Yield: 0.247 g (72%). ³¹P NMR (ppm, CDCl₃): 13.1 (d, J_{PP}=46 Hz), 72.6 (d, J_{PP}=46 Hz).

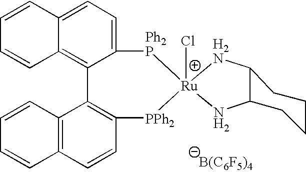

### (i) [RuCl(R-binap)(Ph₂PCH₂CH₂NH₂)][B(C₆F₅)₄]

In an Ar filled flask, 0.200 g (0.20 mmol) of RuCl₂(R-binap)(Ph₂PCH₂CH₂NH₂) and 0.170 g (0.20 mmol) of [Li(OEt₂)₂₅][B(C₆F₅)₄] were combined. CH₂Cl₂ (10 mL) was added and the resulting dark orange suspension was left to stir at ambient temperature for sixteen hours after which time it was filtered, in air, through Celite. The dark orange filtrate was reduced to dryness leaving a deep orange residue. Yield: 0.273 g (84%). ³¹P NMR (ppm, CD₂Cl₂): 31.3 (dd, J_{PP}=31 Hz, J_{PP}=24 Hz), 48.0 (dd, J_{PP}=35 Hz, J_{PP}=31 Hz), 62.2 (dd, J_{PP}=35 Hz, J_{PP}=24 Hz).

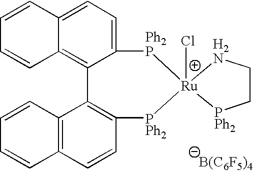

### (j) [RuCl(Ph₂PCH₂CH₂NH₂)₂][B(C₆F₅)₄]

In an Ar filled flask, 0.200 g (0.32 mmol) of RuCl₂(Ph₂PCH₂CH₂NH₂)₂ and 0.276 g (0.32 mmol) of [Li(OEt₂)_{2.5}][B(C₆F₅)₄] were combined. CH₂Cl₂ (10 mL) was added and the resulting orange suspension was left to stir at ambient temperature for sixteen hours after which time it was filtered, in air, through Celite. The orange filtrate was reduced to dryness leaving a deep orange residue. Yield: 0.273 g (86%). ³¹P NMR (ppm, acetone-D₆): 54.9 (d, J_{PP}=36 Hz), 72.7 (d, J_{PP}=36 Hz).

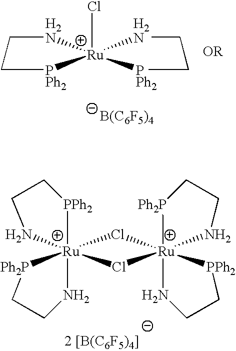

### (k) [RuCl(R-tolbinap)(R,R-Ph₂PCH(Ph)CH(CH₃)NH₂)]BF₄

In an Ar filled flask, 0.150 g (0.13 mmol) of RuCl₂(R-tolbinap)(R,R-Ph₂PCH(Ph)CH(Me)NH₂) and 0.025 g (0.13 mmol) of AgBF₄ were combined. CH₂Cl₂ (10 mL) was added and the resulting brown suspension was left to stir at ambient temperature for 2 hours. The suspension was then filtered, in air, through Celite and the brown filtrate was reduced to dryness leaving a brown residue. Yield: 0.112 g (72%). ³¹P NMR (ppm, CD₂Cl₂): 25.3 (br m), 48.6 (br t, J_{PP}=30 Hz), 87.6 (dd, J_{PP}=30 Hz, J_{PP}=20 Hz).

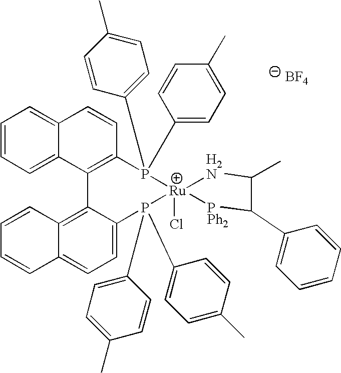

### (l) [RuCl(R-tolbinap)(R,R-Ph₂PCH(Ph)CH(CH₃)NH₂)][B(C₆F₅)₄]

In an Ar filled flask, 0.100 g (0.085 mmol) of RuCl₂(R-tolbinap)(R,R-Ph₂PCH(Ph)CH(Me)NH₂) and 0.074 g (0.085 mmol) of [Li(OEt₂)₂₅][B(C₆F₅)₄] were combined. CH₂Cl₂ (10 mL) was added and the resulting brown suspension was left to stir at ambient temperature for 2 hours. The suspension was then filtered, in air, through Celite and the brown filtrate was reduced to dryness leaving a brown residue. Yield: 0.123 g (79%). ³¹P NMR (ppm, CD₂Cl₂): 25.3 (br m), 48.4 (br t, J_{PP}=30 Hz), 87.7 (dd, J_{PP}=30 Hz, J_{PP}=20 Hz).

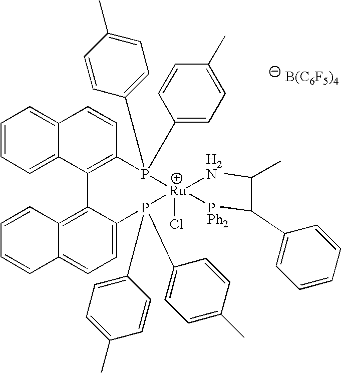

### (m) [RuCl(R-3,5-xylylbinap)(R,R-Ph₂PCH(Ph)CH(CH₃)NH₂)]BF₄

³¹P NMR (ppm, CD₂Cl₂): 31.6 (br m), 46.3 (br t, J_{PP}=32 Hz), 89.4 (br m).

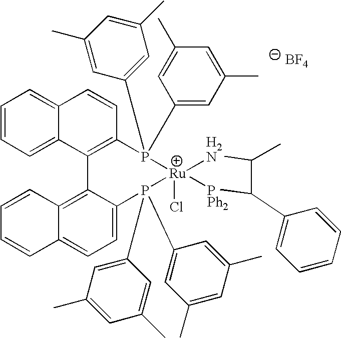

### (n) [RuCl(R-3,5-xylylbinap)(R,R-Ph₂PCH(Ph)CH(CH₃)NH₂)][B(C₆F₅)₄]

³¹P NMR (ppm, CD₂Cl₂): 31.6 (br m), 46.3 (br m), 89.4 (dd, J_{PP}=33 Hz, J_{PP}=28 Hz).

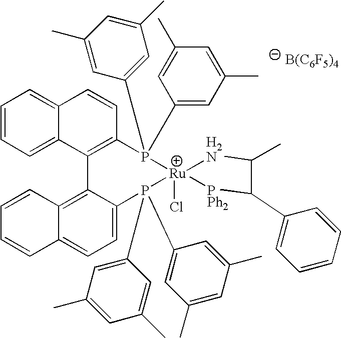

### (o) [RuCl(NH₂CH₂Py)(PPh₃)₂]BF₄

³¹P NMR (ppm, CD₂Cl₂): 14.2 (br), 38.4 (br m), 56.6 (br m).

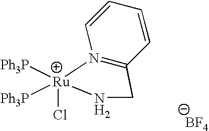

### (p) [RuCl(NH₂CH₂Py)(PPh₃)₂][B(C₆F₅)₄]

³¹P NMR (ppm, CD₂Cl₂): 38.8 (d, J_{PP}=27 Hz), 41.0 (br), 55.4 (d, J_{PP}=27 Hz).

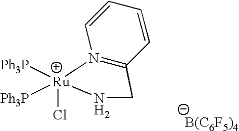

### (q) [RuCl(R-binap)(S_{C},R_{P}—(NH₂CH(CH₃)-Fc-PPh₂))]BF₄

³¹P NMR (ppm, CD₂Cl₂): 42.4 (br m), 54.5 (br).

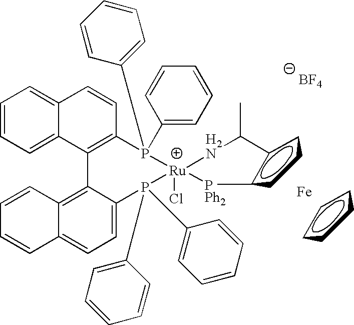

### (r) [RuCl(R-binap)(R_{C},S_{P}—(NH₂CH(CH₃)-Fc-PPh₂))]BF₄

³¹P NMR (ppm, CD₂Cl₂): No resolved peaks at ambient temperature.

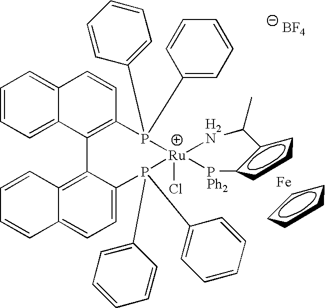

### (s) [RuCl(R-binap)(S_{C},R_{P}—(NH₂CH(CH₃)-Fc-PPh₂))][B(C₆F₅)₄]

³¹P NMR (ppm, CD₂Cl₂): 42.7 (br m), 55.1 (br m).

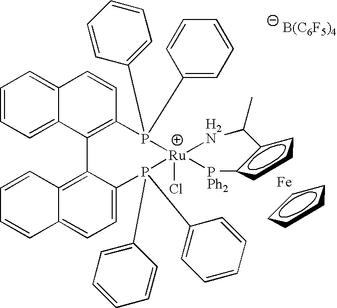

### (t) [RuCl(R-binap)(R_{C},S_{P}—(NH₂CH(CH₃)-Fc-PPh₂))][B(C₆F₅)₄]

³¹P NMR (ppm, CD₂Cl₂): 44.0 (br), 68.0 (br).

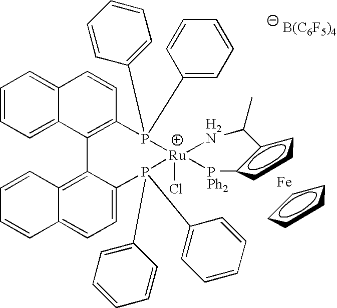

### (u) [RuCl(R,R-DPPcydn)]BF₄

³¹P NMR (ppm, CD₂Cl₂): Several broadened peaks between 10 and 70 ppm.

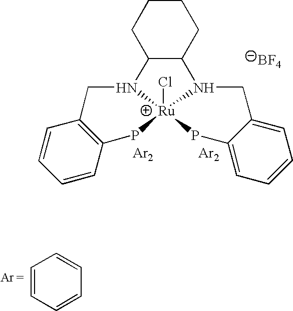

### (v) [RuCl(R,R-DPPcydn)][B(C₆F₅)₄]

³¹P NMR (ppm, CD₂Cl₂): Several broadened peaks between 10 and 70 ppm.

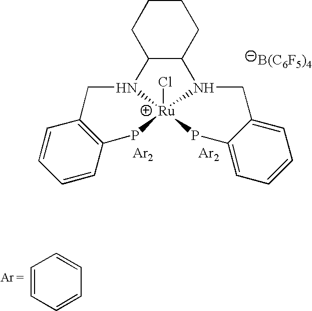

### (w) [RuCl(R,R-di(p-tolyl)PPcydn)]BF₄

³¹P NMR (ppm, CD₂Cl₂): Several broadened peaks between 10 and 70 ppm.

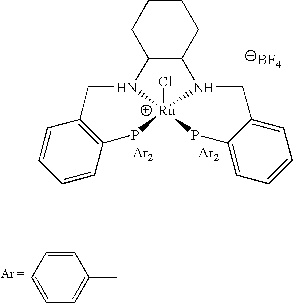

### (x) [RuCl(R,R-di(p-tolyl)PPcydn)][B(C₆F₅)₄]

³¹P NMR (ppm, CD₂Cl₂): Several broadened peaks between 10 and 70 ppm.

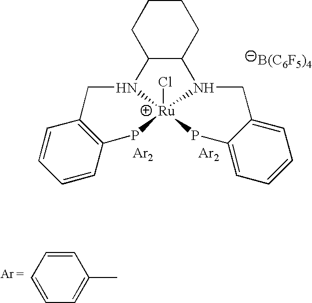

### (y) [RuCl(R,R-di(3,5-xylyl)PPcydn)]BF₄

³¹P NMR (ppm, CD₂Cl₂): Several broadened peaks between 10 and 70 ppm.

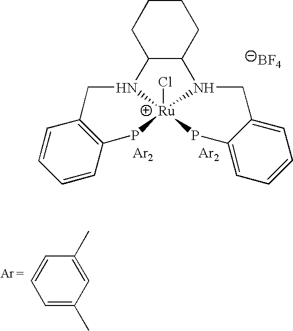

### (z) [RuCl(R,R-di(3,5-xylyl)PPcydn)][B(C₆F₅)₄]

³¹P NMR (ppm, CD₂Cl₂): Several broadened peaks between 10 and 70 ppm.

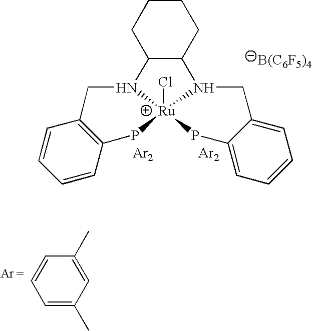

### (aa) [RuCl(S-PPhos)(S-DAIPEN)]BF₄

³¹P NMR (ppm, CD₂Cl₂): Several broadened peaks between 40 and 65 ppm.

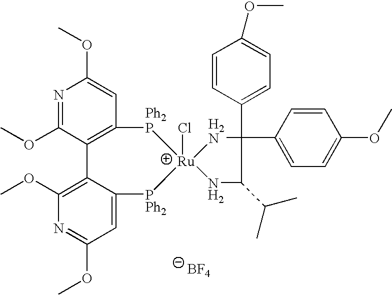

### (bb) [RuCl(S-PPhos)(S-DAIPEN)J][B(C₆F₅)₄]

³¹P NMR (ppm, CD₂Cl₂): Several broadened peaks between 40 and 75 ppm.

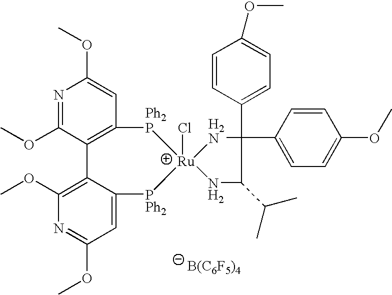

### (cc) [RuCl(S-XylylPPhos)(S-DAIPEN)]BF₄

³¹P NMR (ppm, CD₂Cl₂): Several broadened peaks between 40 and 65 ppm.

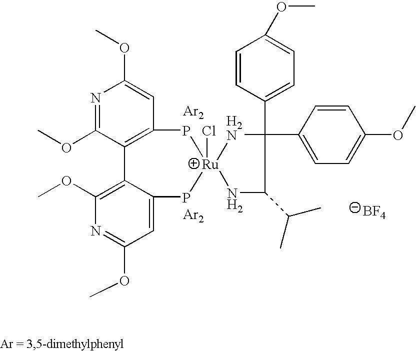

### (dd) [RuCl(S-XylylPPhos)(S-DAIPEN)][B(C₆F₅)₄]

³¹P NMR (ppm, CD₂Cl₂): Several broadened peaks between 40 and 75 ppm.

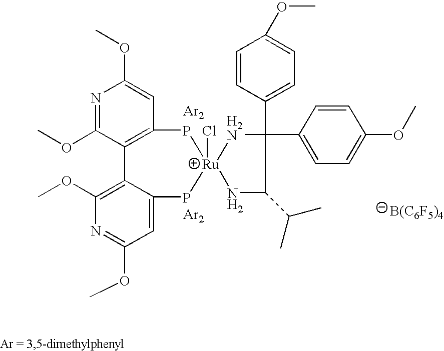

### (ee) [RuCl(S-binap)(S-DAIPEN)]BF₄

³¹P NMR (ppm, CD₂Cl₂): Several broadened peaks between 40 and 65 ppm.

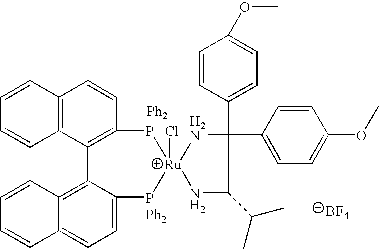

### (ff) [RuCl(S-binap)(S-DAIPEN)][B(C₆F₅)₄]

³¹P NMR (ppm, CD₂Cl₂): Several broadened peaks between −20 and 70 ppm.

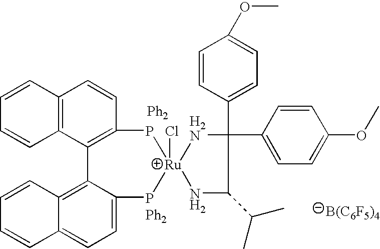

### (gg) [(R-binap)RuCl(R,R-dach)]PF₆.

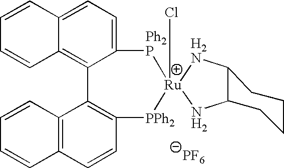

In an Ar filled flask, 0.280 g (0.31 mmol) of RuCl₂(R-binap)(R,R-dach) and 0.078 g (0.31 mmol) of AgPF₆ were combined. CH₂Cl₂ (15 mL) was added and the resulting brown coloured suspension was left to stir at ambient temperature for 24 hours after which time it was filtered, in air, through a 0.45 mm PTFE syringe filter. The orange filtrate was reduced to dryness leaving an orange residue. Yield: 0.280 g (89%). ³¹P{¹H} NMR (ppm, CDCl₃): 7.53 (d, ²J_{PP}=45 Hz), 67.5 (d, ²J_{PP}=45 Hz), 208.6 (septuplet, ²J_{PF}=710 Hz). **R,R-dach=R,R-cydn(hh)[(R-binap)RuCl(R,R-dach)]OTf.

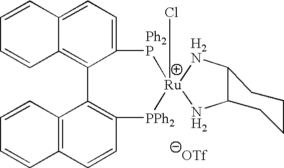

In an Ar filled flask, 0.100 g (0.11 mmol) of RuCl₂(R-binap)(R,R-dach) and 0.028 g (0.11 mmol) of AgOTf were combined. CH₂Cl₂ (5 mL) was added and the resulting rust coloured suspension was left to stir at ambient temperature for 2 hours after which time it was filtered, in air, through a 0.45 mm PTFE syringe filter. The orange filtrate was reduced to dryness leaving an orange residue. Yield: 0.065 g (58%). ³¹P{¹H} NMR (ppm, CDCl₃): 7.53 (d, ²J_{PP}=45 Hz), 67.5 (d, ²J_{PP}=45 Hz).). **R,R-dach =R,R-cydn

### (ii) [(R-binap)RuCl(R,R-dach)][B(3,5-(CF₃)₂C₆H₃)₄]

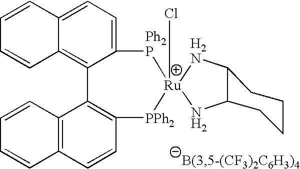

In an Ar filled flask, 0.100 g (0.11 mmol) of RuCl₂(R-binap)(R,R-dach), 0.097 g (0.11 mmol) of Na[B(3,5-(CF₃)₂C₆H₃)₄] and 21 mg (0.11 mmol) of AgBF₄ were combined. CDCl₃ (2 mL) was added and the resulting rust coloured suspension was left to stir at ambient temperature for 18 hours after which time it was filtered, in air, through a 0.45 mm PTFE syringe filter. The orange filtrate was reduced to dryness leaving a yellow-orange residue. Yield: 0.115 g (61%). 31P{¹H} NMR (ppm, CDCl₃): 7.73 (d, ²J_{PP}=45 Hz), 67.2 (d, ²J_{PP}=45 Hz)). **R,R-dach=R,R-cydn

### (jj) [(R-binap)RuCl(PGly)]PF₆

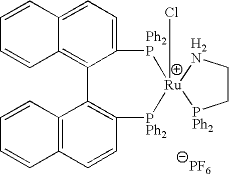

In an Ar filled flask, 0.075 g (0.074 mmol) of RuCl₂(R-binap)(Ph₂PCH₂CH₂NH₂) and 0.019 g (0.074 mmol) of AgPF₆ were combined. CH₂Cl₂ (5 mL) was added and the resulting dark orange suspension was left to stir at ambient temperature for 24 hours after which time it was filtered, in air, through a 0.45 mm PTFE syringe filter. The dark brown-orange filtrate was reduced to dryness leaving a brown residue. Yield: 0.032 g (39%). ³¹P{¹H} NMR (ppm, CDCl₃): 32.6 (dd, ²J_{PP}=31 Hz, ²J_{PP}=24 Hz), 48.0 (dd, ²J_{PP}=34 Hz, ²J_{PP}=31 Hz), 62.7 (dd, ²J_{PP}=34 Hz, ²J_{PP}=24 Hz). There is also formation of another unidentified AB signal in ³¹P NMR: 15.3 (d, ²J_{PP}=17 Hz), 17.3 (d, ²J_{PP}=17 Hz). **PGly=Ph₂PCH₂CH₂NH₂

### (kk) [(R-binap)RuCl(PGly)][B(3,5-(CF₃)₂C₆H₃)₄]

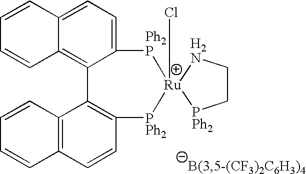

In an Ar filled flask, 0.08 g (0.078 mmol) of RuCl₂(R-binap)(Ph₂PCH₂CH₂NH₂), 0.069 g (0.078 mmol) of Na[B(3,5-(CF₃)₂C₆H₃)₄] and 15 mg (0.078 mmol) of AgBF₄ were combined. CH₂Cl₂ (2 mL) was added and the resulting rust coloured suspension was left to stir at ambient temperature for 18 hours after which time it was filtered, in air, through a 0.45 mm PTFE syringe filter. The orange filtrate was reduced to dryness leaving a yellow-orange residue. Yield: 0.090 g (63%). ³¹P{¹H} NMR (ppm, CDCl₃): 7.73 (d, ²J_{PP}=45 Hz), 67.2 (d, ²J_{PP}=45 Hz). **PGly=Ph₂PCH₂CH₂NH₂

### (ll) [(R-binap)RuCl(PGly)]OTf

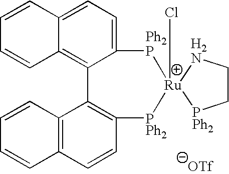

In an Ar filled flask, 0.150 g (0.15 mmol) of RuCl₂(R-binap)(PGly) and 0.038 g (0.15 mmol) of AgOTf were combined. CH₂Cl₂ (5 mL) was added and the resulting dark brown suspension was left to stir at ambient temperature for 24 hours after which time it was filtered, in air, through a 0.45 mm PTFE syringe filter. The dark brown filtrate was reduced to dryness leaving a brown-yellow residue. Yield: 0.130 g (78%). ³¹P{¹H} NMR (ppm, CDCl₃): 29.4 (t, ²J_{PP}=27 Hz), 45.8 (dd, ²J_{PP}=33 Hz), 60.5 (t, ²J_{PP}=27 Hz). There is also formation of another unidentified AB signal in ³¹P NMR: 13.4 (d, ²J_{PP}=17 Hz), 15.4 (d, ²J_{PP}=17 Hz). **PGly=Ph₂PCH₂CH₂NH₂

### (mm) [RuCl(PGly)₂]PF₆

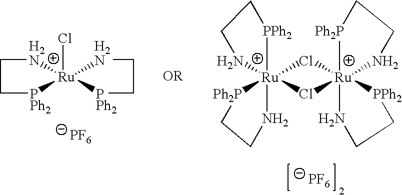

In an Ar filled flask, 0.075 g (0.12 mmol) of RuCl₂(PGly)₂ and 0.030 g (0.12 mmol) of AgPF₆ were combined. CH₂Cl₂ (5 mL) was added and the resulting brown suspension was left to stir at ambient temperature for 24 hours after which time it was filtered, in air, through a 0.45 mm PTFE syringe filter. The dark orange filtrate was reduced to dryness leaving a yellow brown residue. Yield: 0.030 g (35%). ³¹P{¹H} NMR (ppm, CDCl₃): 7 different doublets in the range 52-73 ppm. **PGly=Ph₂PCH₂CH₂NH₂

### (nn) [RuCl(PGly)₂]OTf

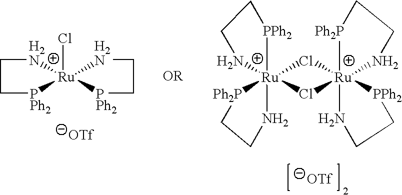

In an Ar filled flask, 0.150 g (0.24 mmol) of RuCl₂(PGly)₂ and 0.061 g (0.24 mmol) of AgPF₆ were combined. CH₂Cl₂ (10 mL) was added and the resulting brown suspension was left to stir at ambient temperature for 24 hours after which time it was filtered, in air, through a 0.45 mm PTFE syringe filter. The dark yellow brown filtrate was reduced to dryness leaving an orange residue. Yield: 0.095 g (53%). ³¹P{¹H} NMR (ppm, CDCl₃): 7 different doublets in the range 52-73 ppm. **PGly=Ph₂PCH₂CH₂NH₂

### (oo) [RuCl(PGly)₂][B(3,5-(CF₃)₂C₆H₃)₄]

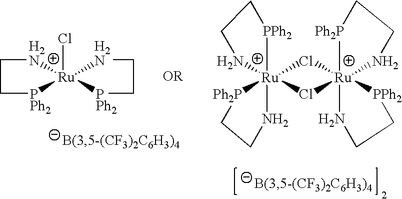

In an Ar filled flask, 0.08 g (0.13 mmol) of RuCl₂(PGly)₂, 0.112 g (0.13 mmol) of Na[B(3,5-(CF₃)₂C₆H₃)₄] and 25 mg (0.13 mmol) of AgBF₄ were combined. CH₂Cl₂ (2 mL) was added and the resulting rust coloured suspension was left to stir at ambient temperature for 18 hours after which time it was filtered, in air, through a 0.45 mm PTFE syringe filter. The orange filtrate was reduced to dryness leaving an orange residue. Yield: 0.030 g (16%). 6 different doublets in the range 30-51 ppm. **PGly=Ph₂PCH₂CH₂NH₂

### (pp) [RuCl(S-PhanePhos)(R,R-DPEN)]BF₄

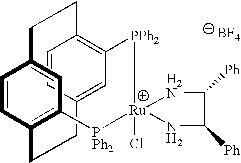

In an Ar filled flask, 0.100 g (0.10 mmol) of RuCl₂(S-PhanePhos)(R,R-DPEN) and 0.020 g (0.10 mmol) of AgBF₄ were combined. CH₂Cl₂ (5 mL) was added and the resulting brown suspension was left to stir at ambient temperature for sixteen hours. The suspension was filtered, in air, through Celite and the brown filtrate was reduced to dryness leaving a brown residue. Yield: 0.048 g (46%). ³¹P NMR (ppm, CD₂Cl₂): 52.0 (d, J_{PP}=28 Hz), 43.1 (d, J_{PP}=28 Hz).

### (qq) [RuCl(S-PhanePhos)(R,R-DPEN)]B(C₆F₅)₄

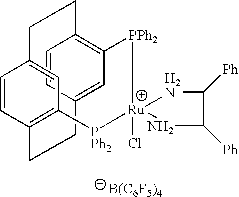

In an Ar filled flask, 0.050 g (0.06 mmol) of RuCl₂(S-PhanePhos)(R,R-DPEN) and 0.045 g (0.06 mmol) of Li(OEt₂)_{2.5}[B(C₆F₅)₄] were combined. CH₂Cl₂ (5 mL) was added and the resulting brown suspension was left to stir at ambient temperature for sixteen hours. The suspension was filtered, in air, through Celite and the brown filtrate was reduced to dryness leaving a brown residue. Yield: 0.036 g (40%). ³¹P NMR (ppm, CD₂Cl₂): 50.5 (d, J_{PP}=28 Hz), 42.4 (d, J_{PP}=28 Hz).

### (rr) [RuCl(S-XylylPhanePhos)(R,R-DPEN)]BF₄

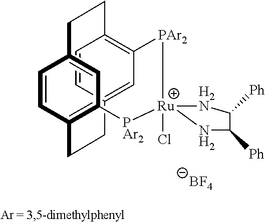

In an Ar filled flask, 0.100 g (0.10 mmol) of RuCl₂(S-XylylPhanePhos)(R,R-DPEN) and 0.018 g (0.10 mmol) of AgBF₄ were combined. CH₂Cl₂ (5 mL) was added and the resulting brown suspension was left to stir at ambient temperature for sixteen hours. The suspension was filtered, in air, through Celite and the brown filtrate was reduced to dryness leaving a brown residue. Yield: 0.066 g (63%). ³¹P NMR (ppm, CD₂Cl₂): 52 (d, J_{PP}=28 Hz), 42 (d, J_{PP}=28 Hz).

### (ss) [RuCl(S-XylylPhanePhos)(R,R-DPEN)]B(C₆F₅)₄

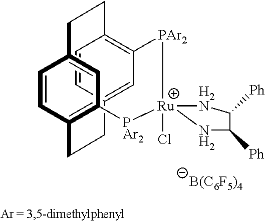

In an Ar filled flask, 0.050 g (0.05 mmol) of RuCl₂(S-PhanePhos)(R,R-DPEN) and 0.041 g (0.05 mmol) of Li(OEt₂)₂₅[B(C₆F₅)₄] were combined. CH₂Cl₂ (5 mL) was added and the resulting brown suspension was left to stir at ambient temperature for sixteen hours. The suspension was filtered, in air, through Celite and the brown filtrate was reduced to dryness leaving a brown residue. Yield: 0.065 g (81%). ³¹P NMR (ppm, CD₂Cl₂): 52.2 (d, J_{PP}=29 Hz), 41.5 (d, J_{PP}=29 Hz).

### (tt) [RuCl(PPh₃)₂((S)-1-(pyridin-2-yl)ethanamine)]BF₄

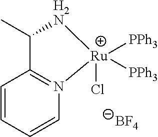

In an Ar filled flask, 0.100 g (0.12 mmol) of RuCl₂(PPh₃)₂((S)-1-(pyridin-2-yl)ethanamine) and 0.024 g (0.12 mmol) of AgBF₄ were combined. CH₂Cl₂ (5 mL) was added and the resulting brown suspension was left to stir at ambient temperature for two hours. The suspension was filtered through a 0.45 μm PTFE syringe filter and the brown filtrate was reduced to dryness leaving a brown residue. Yield: 0.070 g (66%). ¹⁹F NMR (282 MHz, CD₂Cl₂): −152 (s).

### (uu) [RuCl(S-XylylPPhos)((S)-1-(pyridin-2-yl)ethanamine)]BF₄

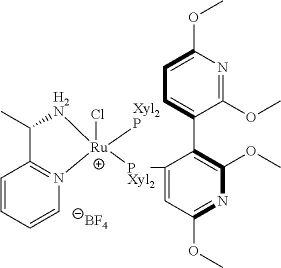

In an Ar filled flask, 0.150 g (0.14 mmol) of RuCl₂(S-XylylPPhos)((S)-1-(pyridin-2-yl)ethanamine) and 0.027 g (0.14 mmol) of AgBF₄ were combined. CH₂Cl₂ (5 mL) was added and the resulting brown suspension was left to stir at ambient temperature for two hours. The suspension was filtered through a 0.45 μm PTFE syringe filter and the brown filtrate was reduced to dryness leaving a brown residue. Yield: 0.127 g (81%). ¹⁹F NMR (282 MHz, CD₂Cl₂): −152 (s).

### (vv) [RuCl(R-BINAP)((S)-1-(pyridin-2-yl)ethanamine)]BF₄

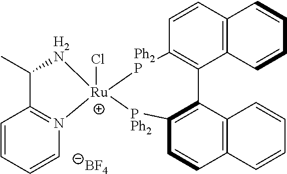

In an Ar filled flask, 0.150 g (0.16 mmol) of RuCl₂(R-BINAP)((S)-1-(pyridin-2-yl)ethanamine) and 0.032 g (0.16 mmol) of AgBF₄ were combined. CH₂Cl₂ (5 mL) was added and the resulting brown suspension was left to stir at ambient temperature for two hours. The suspension was filtered through a 0.45 μm PTFE syringe filter and the brown filtrate was reduced to dryness leaving a brown residue. Yield: 0.087 g (55%). ³¹P NMR (ppm, CD₂Cl₂): No resolved peaks at ambient temperature. ¹⁹F NMR (282 MHz, CD₂Cl₂): −152 (s).

### (ww) [RuCl(S_{C},R_{P}-PCy₂-CH(CH₃)-Fc-PCy₂)((S)-1-(pyridin-2-yl)ethanamine)]BF₄

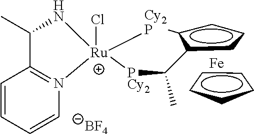

In an Ar filled flask, 0.075 g (0.08 mmol) of RuCl₂(S_{C},R_{P}-PCy₂-CH(CH₃)-Fc-PCy₂)((S)-1-(pyridin-2-yl)ethanamine) and 0.016 g (0.08 mmol) of AgBF₄ were combined. CH₂Cl₂ (5 mL) was added and the resulting brown suspension was left to stir at ambient temperature for two hours. The suspension was filtered through a 0.45 μM PTFE syringe filter and the brown filtrate was reduced to dryness leaving a brown residue. Yield: 0.079 g (63%). ¹⁹F NMR (282 MHz, CD₂Cl₂): −152 (s).

### (xx) [RuCl(R-binap)(R,R-cydn)]CB₁₂H₁₂

In the dry box, RuCl₂(Binap)(cydn) (0.18 g, 0.19 mmol) was dissolved in CH₂Cl₂ and Ag(CB₁₁H₁₂) (50 mg, 0.19 mmol) was dissolved in benzene and CH₂Cl₂. The two portions were then mixed and stirred for half hour. The AgCl then formed was filtered off and the compound was recrystallized from hexanes. Yield: 0.15 g, 74%.

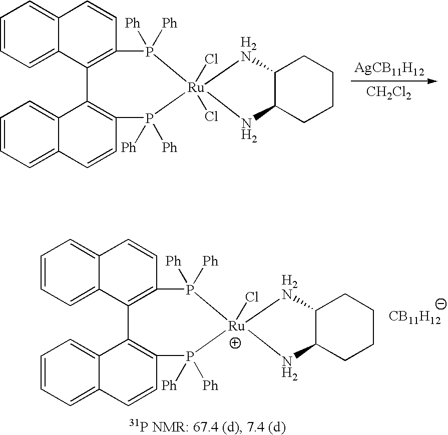

### (yy) [RuCl(R-binap)(R,R-cydn)]CB₁₂H₆Br₆

In the dry box, RuCl₂(Binap)(cydn) (12.5 mg, 0.014 mmol) was dissolved in CH₂Cl₂ and Ag(CB₁₁H₆Br₆) (10 mg, 0.014 mmol) was dissolved in benzene and CH₂Cl₂. The two portions were then mixed and stirred for half hour. The AgCl then formed was filtered off and the compound was recrystallized from hexanes. Yield: 15 mg, 73%.

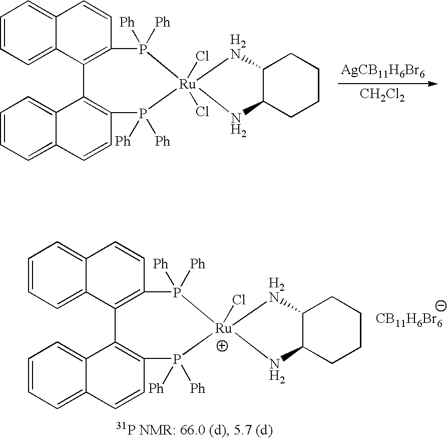

### Example 3

### Alternate Route to Cationic Ruthenium Hydrogenation Catalysts

In the syntheses described above, the neutral precursor complexes were treated with anion abstracting agents to render the complexes cationic. The neutral precursors were generally derived from the ubiquitous ruthenium compounds [RuCl₂(p-cymene)]₂, [RuCl₂(benzene)]₂ or [RuCl₂(cod)]ₙ (cod=cyclooctadiene). These are common synthons used to prepare a range of ruthenium complexes and are known to be notoriously insoluble materials. As a result of the insolubility of these complexes, the preparation of Ru derivatives from these material require long reaction times and forcing conditions.

An alternate route to the same cationic ruthenium hydrogenation catalysts exists in the use of a cationic Ru precursor. Indeed, a cationic derivative of [RuCl₂(p-cymene)]₂ holds the promise of improved solubility and thus shorter reaction times and less forcing conditions. To this end, the reaction of [RuCl₂(p-cymene)]₂ with anion abstracting agents was explored and found to yield the desired cationic synthon according to Scheme 2 below. The complexes [Ru₂Cl₃(p-cymene)₂][PF₆] and [Ru(MeCN)₃(p-cymene)]₂[BF₄]₂ were described by Bennett et al., J. C. S. Dalton Trans. 1974, 233. Limited synthetic and spectroscopic details were provided in this report.

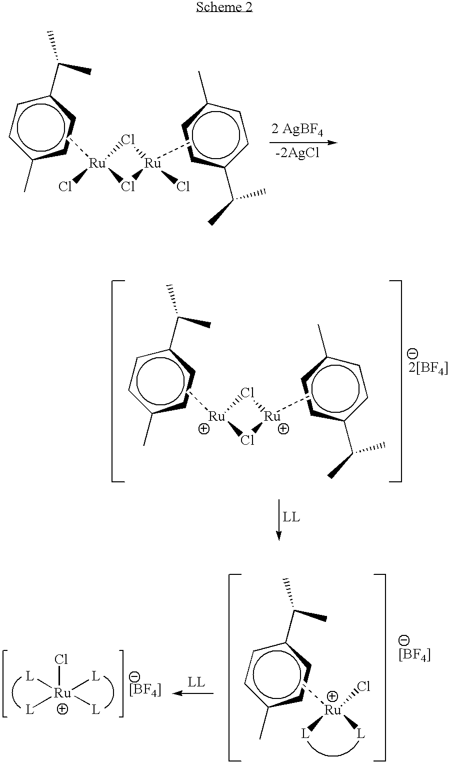

### (a) [RuCl(p-cymene)]₂[BF₄]₂

In an Ar filled flask, 0.25 g (0.041 mmol) [RuCl₂(p-cymene)]₂ and 0.16 g (0.082 mmol) of AgBF₄ were combined. CH₂Cl₂ (10 mL) was added and the resulting orange suspension was left to stir at ambient temperature. Within several minutes the suspension darkened to brown/green in colour. After 2 hours, the suspension was filtered through Celite and the orange filtrate was reduced to approximately 1 mL in volume. Addition of hexane afforded an oily orange solid which was washed repeatedly with hexane and dried in vacuo. Yield: 0.215 g (74%).

### (b) In Situ Preparation of Cationic Ruthenium Hydrogenation Catalyst

In an Ar filled flask, 0.070 g (0.11 mmol) [RuCl₂(p-cymene)]₂ and 0.045 g (0.11 mmol) of AgBF₄ were combined. CH₂Cl₂ (10 mL) was added and the resulting orange suspension was left to stir at ambient temperature. Within several minutes the suspension darkened to brown/green in colour. After 2 hours, the suspension was filtered through Celite and the orange filtrate was collected and set to stir. A solution of R-BINAP (0.142 g, 0.11 mmol) in toluene (5 mL) was added. The resulting solution was stirred for several minutes. Solid R,R-cydn (0.026 g, 0.11 mmol) was added. The resulting solution was heated to 60° C. for approximately 3 hours. The resulting solution was concentrated to dryness leaving an orange residue. A sample of the residue was employed in the catalytic hydrogenation of acetophenone according to the conditions described below. Result: time=2 h; conv.=>99%; ee=84%.

### Example 4

### Second Alternate Route to Cationic Ruthenium Hydrogenation Catalysts

Yet another route exists via an ill-defined mixture of ruthenium-diphosphine-DMF complexes (DMF=dimethylformamide) reported in the literature (Noyori et al., Tetrahedron Letters, 1991, 32, 4163). The mixture is believed to consist of the following components: RuCl₂(diphosphine)(DMF)₂ and [RuCl₂(diphosphine)(DMF)]ₙ). Thus, treatment of the RuCl₂(BINAP)(DMF)₂ and [RuCl₂(BINAP)(DMF)]ₙ) mixture with an equivalent of an anion abstracting agent (e.g. AgBF₄) to generate a cationic precursor which then react with a diamine (e.g. R,R-cydn) to yield the cationic ruthenium-diphosphine-diamine complex, [RuCl(R-BINAP)(R,R-cydn)]BF₄. This synthetic route is presented in Scheme 3 below. It should be noted that the product of this reaction also appears to be a mixture of the DMF-coordinated cation and the DMF-free cation.

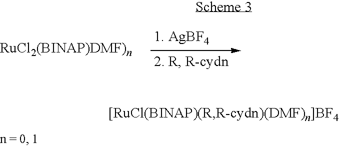

This procedure can also be applied to the synthesis of compounds (I), (III) and (V) of this disclosure.

### (a) [RuCl₂(R-BINAP)DMF)ₙ]

In an Ar filled flask, 0.250 g (0.50 mmol) of [RuCl₂(C₆H₆)]₂ and 0.622 g (1.00 mmol) of R-BINAP were combined. DMF (5 mL) was added and the resulting brown suspension was set to stir in a 100° C. oil bath. After 15 minutes, the suspension had cleared to a red/brown solution. The flask was removed from the oil bath and allowed to cool to RT. The solution was then concentrated to an oily residue and Et₂O (20 mL) was added affording brick red solids. The solids were filtered off in air, washed with Et₂O (5×5 mL) and dried in vacuo. Yield: 0.820 g (87%). ³¹P NMR (ppm, CD₂Cl₂): several broad doublets between 50-62 ppm.

### (b) [RuCl(R-BINAP)(R,R-cydn)(DMF)ₙ]BF₄

In an Ar filled flask, 0.200 g (0.21 mmol) of RuCl₂(R-BINAP)(DMF)₂ and 0.041 g (0.21 mmol) of AgBF₄ were combined. CH₂Cl₂ (10 mL) was added and the resulting brown suspension was set to stir at ambient temperature. After 2 hours, 0.024 g (0.21 mmol) of R,R-cydn in CH₂Cl₂ (1 mL) was added and the suspension quickly changed to green in colour. The suspension was stirred for a further 2 hours and then filtered through a 0.45 mm PTFE syringe filter. The green filtrate was concentrated to approximately 1 mL and Et₂O (20 mL) was added affording green solids. The solids were filtered off in air, washed with Et₂O (4×5 mL) and dried in vacuo. Yield: 0.186 g (85%). ³¹P NMR (ppm, CD₂Cl₂): 7.38 (d, J_{PP}=45 Hz), 67.4 (d, J_{PP}=45 Hz). These chemical shift values match those for the same compound prepared via treatment of RuCl₂(R-BINAP)(R,R-cydn) with one equivalent of AgBF₄. Minor peaks are also present between 48-54 ppm which are consistent with the presence of a small amount of a DMF adduct of the form “[RuCl(R-BINAP)(R,R-cydn)(DMF)]BF₄” which would account for the green colour (vs. orange for the same material prepared via treatment of RuCl₂(R-BINAP)(R,R-cydn) with AgBF₄).

### (c) [RuCl(R-BINAP)(R,R-cydn)(DMF)ₙ]B(C₆F₅)₄

In an Ar filled flask, 0.200 g (0.21 mmol) of RuCl₂(R-BINAP)(DMF)₂ and 0.185 g (0.21 mmol) of Li(OEt₂)_{2.5}[B(C₆F₅)₄] were combined. CH₂Cl₂ (10 mL) was added and the resulting brown suspension was set to stir at ambient temperature. After 2 hours, 0.024 g (0.21 mmol) of R,R-cydn in CH₂Cl₂ (1 mL) was added and the suspension gradually changed to green in colour. The suspension was stirred for a further 2 hours and then filtered through a 0.45 mm PTFE syringe filter. The green filtrate was concentrated to approximately 1 mL and hexane (20 mL) was added affording green solids. The solids were filtered off in air, washed with hexane (4×5 mL) and dried in vacuo. Yield: 0.333 g (96%). ³¹P NMR (ppm, CD₂Cl₂): 7.31 (d, J_{PP}=45 Hz), 67.4 (d, J_{PP}=45 Hz). These chemical shift values match those for the same compound prepared via treatment of RuCl₂(R-binap)(R,R-cydn) with one equivalent of Li(OEt₂)_{2.5}[B(C₆F₅)₄]. Minor peaks are also present between 52-54 ppm which are consistent with a dmf adduct of the form “[RuCl(R-BINAP)(R,R-cydn)(DMF)]B(C₆F₅)₄” which would account for the green colour (vs. orange for the same material prepared via treatment of RuCl₂(R-BINAP)(R,R-cydn) with Li(OEt₂)_{2.5}[B(C₆F₅)₄]).

### Example 5

### Third Alternate Route to Cationic Ruthenium Hydrogenation Catalysts

Another route to a cationic ruthenium catalyst exists through the stable precursor RuCl₂(diphosphine)(pyridine)₂. It has been determined that RuCl₂(diphosphine)(pyridine)₂ is a highly useful and convenient precursor to complexes of the type [RuCl(diphosphine)(diamine)LB]X and [RuCl(diphosphine)-(aminophosphine)LB]X. The precursor, RuCl₂(diphosphine)(pyridine)₂ is a well defined, single component (in contrast to the DMF analogue of Example 4). RuCl₂(diphosphine)(pyridine)₂ can be prepared from the corresponding DMF complex or in an analogous method to the preparation for the DMF complex wherein pyridine is used instead of DMF, as shown in Scheme 4.

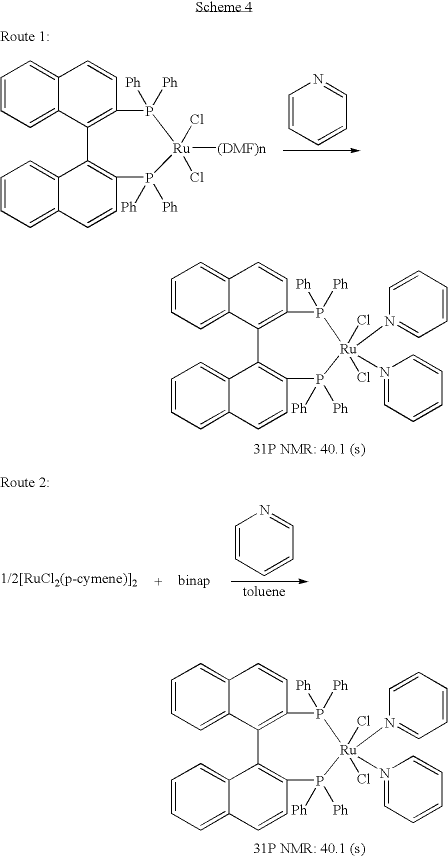

The precursor compound RuCl₂(diphosphine)(pyridine)₂ is readily derivatized into its cationic counterpart, [RuCl(diphopshine)(pyridine)₂]BF₄, by treatment with an anion abstracting agent (e.g. AgBF₄ as set out in Scheme 5).

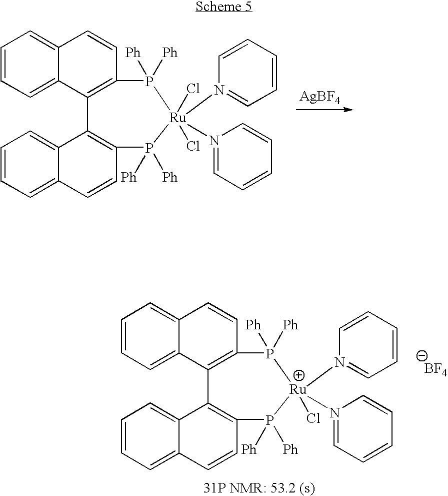

The cation, an air stable solid which can be isolated in high yields and stored under ambient conditions, is a convenient precursor to other cationic hydrogenation catalysts. The cationic pyridine compound can be derivatized by treatment with a diamine into compounds of the type [RuCl(diphosphine)(diamine)]BF₄ (Examples 5(a) and (b)).

An alternate route to complexes of the type [RuCl(diphosphine)(pyridine)₂]⁺ via a ruthenium-norbornadiene (NBD) complex which is equally valuable is outlined below in Scheme 6. It should be noted that pyridine can be replaced by any Lewis base and the product can be further derivatized to complexes of the type [RuCl(diphosphine)(diamine)LB]X and [RuCl(diphosphine)(aminophosphine)LB]X (where LB is Lewis base).

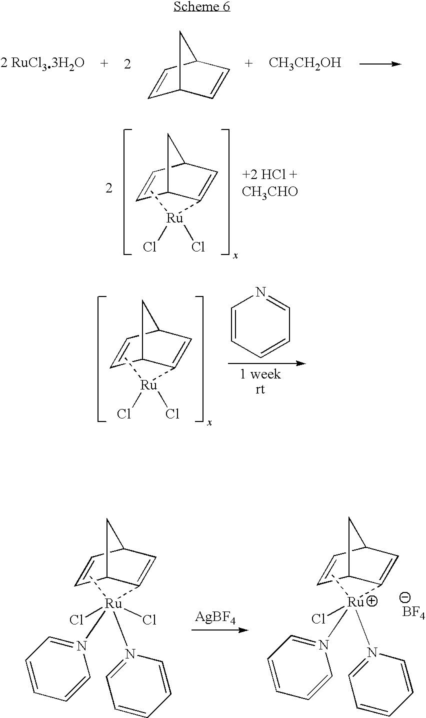

The procedures described in this Example can be generalized into the following method for the preparation of cationic or dicationic catalysts:

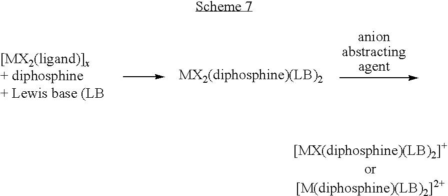

wherein M, X and LB are as defined for the compounds of the disclosure and diphosphine is a P2 ligand as defined for the compounds of the disclosure and ligand is a neutral displaceable ligand such as p-cymene, benzene, COD and NBD and x is an integer that depends on the structure of the complex (typically x is 2).

The cationic catalysts derived from the precursors described in this Example have been tested in hydrogenation using identical procedures as for the cations derived from treatment of the RuCl₂(diphosphine)(diamine) or RuCl₂(diphosphine)(PN) complexes with anion abstracting agents in the presence of Lewis bases. The complexes prepared via the [RuCl(PP)(py)₂]BF₄ precursor display essentially identical behavior in hydrogenation of acetophenone.

### (a) [RuCl(R-binap)(R,R-cydn)(py)]BF₄

To a CH₂Cl₂ solution of [RuCl(R-binap)(py)₂]BF₄ (0.08 g, 0.0796 mmol) was added a CH₂Cl₂ solution of the R-R-cydn (9.1 mg, 0.0796 mmol) under inert (Ar) atmosphere. The reaction mixture was allowed to stir overnight at ambient temperature. The solution was then concentrated, and the residue was recrystallized from CH₂Cl₂/Et₂O. The solid that precipitated was then filtered in air to obtain an amber-yellow color solid. Yield: 0.06 g, 70%. This catalyst was examined for its catalytic ability to convert acetophenone to its corresponding alcohol, and showed a 98% conversion with an enantiomeric excess of 80%.

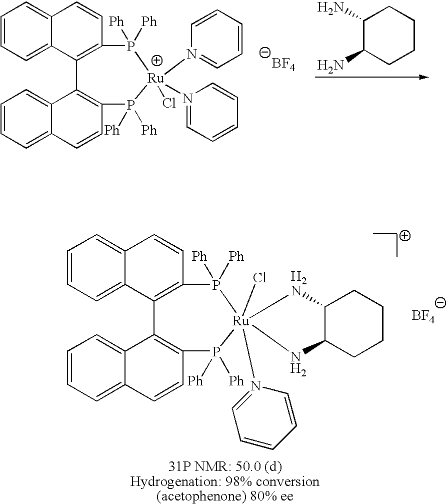

### (b) [RuCl(R-binap)(Ph₂PCH₂CH₂NH₂)(py)]BF₄

To a CH₂Cl₂ solution of the [RuCl(R-binap)(py)₂]BF₄ (0.077 g, 0.0770 mmol) was added a CH₂Cl₂ solution of 2-(diphenylphosphino)ethylamine (17.6 mg, 0.0770 mmol) under inert atmosphere. The reaction mixture was allowed to stir overnight at ambient temperature. During this time some precipitate formed. The solution was then filtered, the filtrate was concentrated, and the residue was recrystallized from CH₂Cl₂/Et₂O. The solid that precipitated was then filtered in air to obtain an amber-yellow color solid. Yield: 0.05 g, 54%. This catalyst was examined for its catalytic ability to convert acetophenone to its corresponding alcohol, and showed a 72% conversion and an enantiomeric excess of 22%.

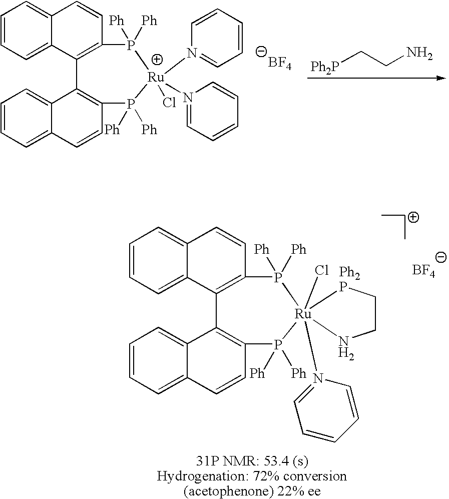

### (c) [RuCl(NBD)(py)₂]BF₄ (As per Scheme 6 above)

The first step of the reaction is carried out in air. To a 500 mL schlenk flask containing a pear-shaped stirring bar is charged with a ethanol solution (200 mL) of RuCl₃.3H₂O, and bicycle[2.2.1]hepta-2,5-diene(norbornadiene) (10 mL, 0.12 mol). The mixture is vigorously stirred at room temperature for 24 hour. During this time the brick red to brown solid precipitated from the solution. On completion of the reaction the suspension is filtered using a medium porosity glass filter frit and washed thoroughly with acetone (50 mL). Drying of the solid gives 3.8 g of insoluble brick red solid. (ref Inorganic Syntheses. New York: John Wiley and Sons, 1989: 250-251)

The second step of the reaction is carried out under Argon and the work-up procedure was slightly modified from the original literature. [(NBD)RuCl₂]ₓ (2.0 g, 7.57 mmol) was rapidly stirred in pyridine (50 mL) for 1 week at room temperature under argon. The mixture changed from brown to greenish-yellow over this period. The pyridine was then removed under vacuum to give a greenish yellow solid. The solid was then dissolved in CH₂Cl₂ and the insoluble black material was filtered off. The CH₂Cl₂ solution was then concentrated, and recrystallized from hexanes 2 times, yielding a dark-orange crystalline materials. Yield: 3.0 (93%). ¹H NMR (400 MHz, CD₂Cl₂): d 1.55 (br s, 2H, CH₂), 4.05 (br s, 2H, bridgehead CH), 4.85 (m, 4H, olefin), 7.25 (br t, J=11.9 Hz, 4H), 7.7 (br t, J=11.9 Hz, 2H), 8.54 (br d, J=12.0 Hz, 4H).(ref Chirality 2000, 12: 514-522)

The third step of the reaction is carried out in the dry box. To a small vial is charged (NBD)RuCl₂(Pyridine)2 (0.1 g, 0.23 mmol) and 1 equiv. of AgBF₄ (46 mg, 0.23 mmol) and CH₂Cl₂ (5 mL). The solution was allowed to stir for 1 hour. Precipitate was observed during this period. The precipitate was then filtered off, and the filtrate was concentrated and recrystallized from Et₂O to obtain a pale greenish-yellow solid. Yield: 80 mg, 72%.

### Example 6

### Synthesis of Dicationic Ruthenium Hydrogenation Catalysts

### (a) [Ru(R-binap)(R, R-cydn)][BF₄]₂

In an Ar filled flask, 0.100 g (0.11 mmol) of RuCl₂(R-binap)(R,R-cydn) and 0.045 g (0.24 mmol) of AgBF₄ were combined. CH₂Cl₂ (7 mL) was added and the resulting rust coloured suspension was left to stir at ambient temperature for two hours after which time it was filtered through Celite. The orange filtrate was reduced to dryness leaving a yellow/orange residue. Yield: 0.085 g (77%). ³¹P NMR (ppm, CD₂Cl₂): 0.48 (d, J_{PP}=39 Hz), 64.89 (d, J_{PP}=39 Hz).

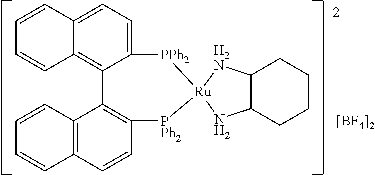

### (b) [Ru (R-binap)(Ph₂PCH₂CH₂NH₂)][BF₄]₂

In an Ar filled flask, 0.115 g (0.11 mmol) of RuCl₂(R-binap)(Ph₂PCH₂CH₂NH₂) and 0.044 g (0.24 mmol) of AgBF₄ were combined. CH₂Cl₂ (7 mL) was added and the resulting dark orange suspension was left to stir at ambient temperature for two hours after which time it was filtered through Celite. The yellow filtrate was concentrated to approximately 1 mL in volume and Et₂O was added (10 mL) affording pale yellow solids. The solids were filtered off, washed with Et₂O (3×5 mL) and dried in vacuo. Yield: 0.119 g (94%). ³¹P NMR (ppm, CD₂Cl₂): 15.3 (d, J_{PP}=18 Hz), 17.2 (d, J_{PP}=18 Hz), 62.2 (br m).

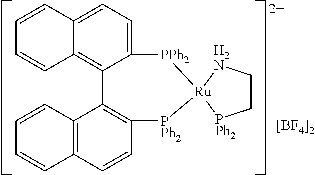

### (c) [Ru(R,R-DPPcydn)][BF₄]₂

In an Ar filled flask, 0.200 g (0.20 mmol) of RuCl₂(R,R-DPPcydn) and 0.093 g (0.48 mmol) of AgBF₄ were combined. CH₂Cl₂ (7 mL) was added and the resulting dark yellow/green suspension was left to stir at ambient temperature for two hours after which time it was filtered through Celite. The yellow filtrate was concentrated to approximately 1 mL in volume and Et₂O was added (10 mL) affording pale yellow solids. The solids were filtered off, washed with Et₂O (3×5 mL) and dried in vacuo. Yield: 0.169 g (92%). ³¹P NMR (ₚₚm, CD₂Cl₂): broad signals at 42.2 and 63.3 ppm barely discernable above baseline.

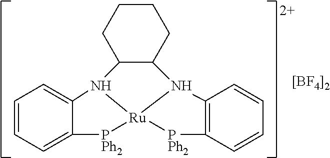

### Example 7

### Lewis Base Adducts of Dicationic Catalysts

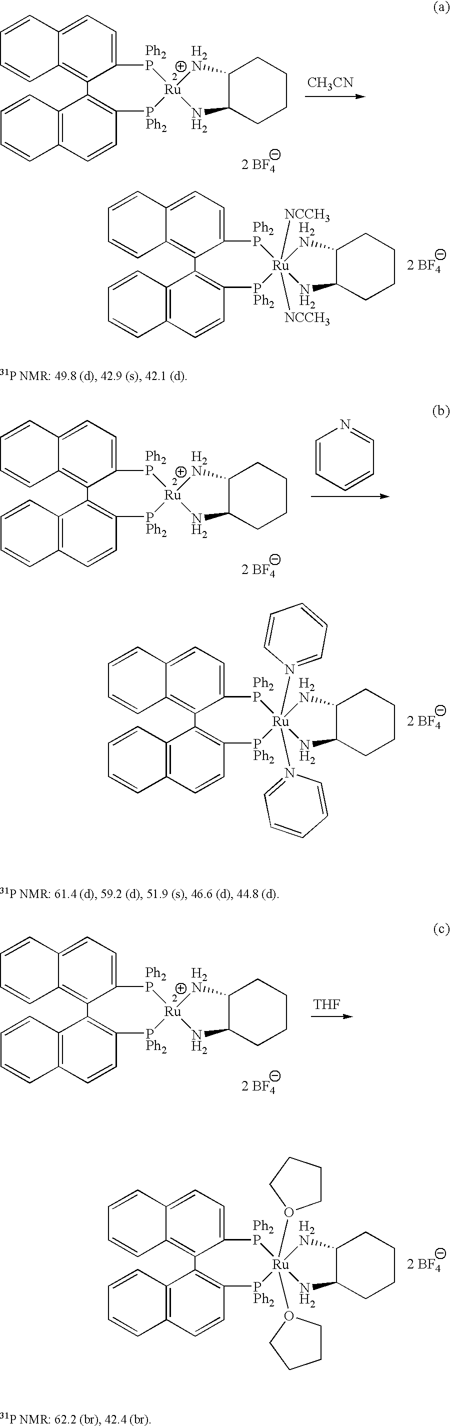

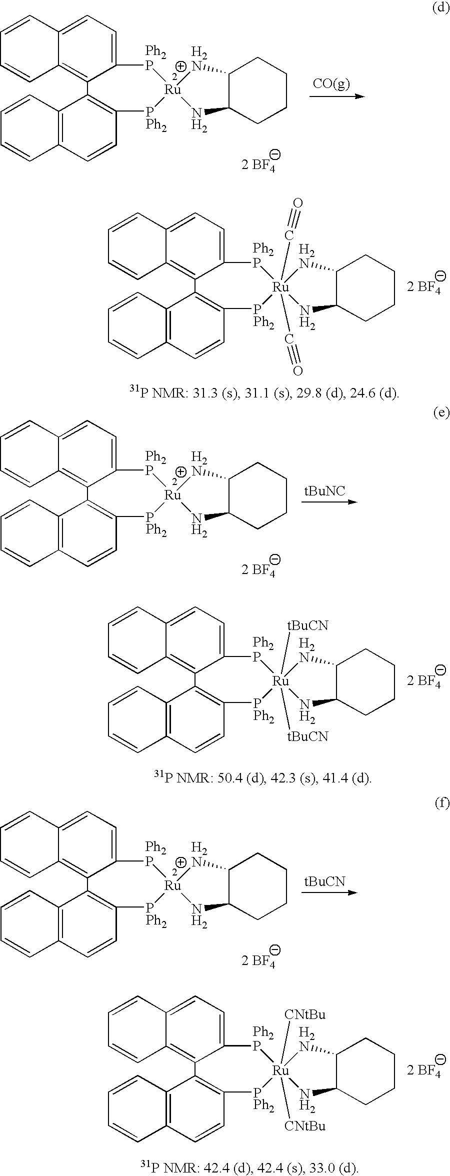

### Example 8

### Cationic Iron Hydrogenation Catalysts

### (a) Bis(acetonitrile)N¹,N²-bis(2-(diphenylphosphino)benzylidene)(R,R)-cyclohexane-1,2-diamine iron(II)tetrafluoroborate, [Fe(NCMe)₂((R,R)-cyPPh₂N₂)][BF₄]₂,

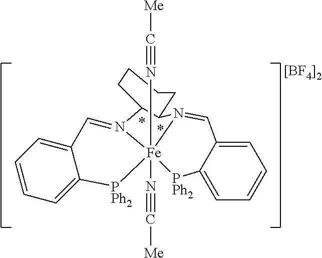

Acetonitrile (5 mL) was added to 210 mg (0.319 mmol) of N¹,N²-bis(2-(diphenylphosphino)benzylidene)cyclohexane-1,2-diamine, (R,R)-cyP₂N₂ and 102 mg (0.302 mmol) of iron(II)tetrafluoroborate hexahydrate[Fe(OH₂)₆][BF₄]₂ and the mixture was stirred for one hour. The solution was concentrated to ca. 1 mL and then 20 mL of diethyl ether was added dropwise. The mixture was stirred for 30 minutes and then the solid was collected on a glass frit and dried in vacuo. Yield: 240 mg, 82%. ³¹P{¹H} NMR (121 MHz, CD₃CN): 52.7 ppm.

### (b) Bis(acetonitrile)N¹,N²-bis(2-(ditolylphosphino)benzyl)(R,R)-cyclohexane-1,2-diamine iron(II)tetrafluoroborate, [Fe(NCMe)₂((R,R)-cyPAr₂(NH)₂)][BF₄]₂

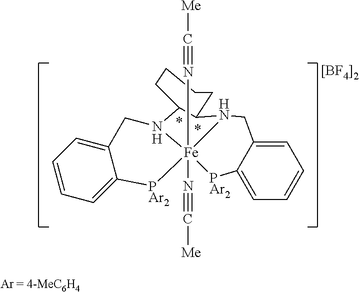

A solution of N¹,N²-bis(2-(ditolylphosphino)benzyl)(R,R)-cyclohexane-1,2-diamine(R,R)-cyPAr₂(NH)₂ (149 mg, 0.207 mmol) and iron(II)tetrafluoroborate hexahydrate[Fe(OH₂)₆][BF₄]₂ (70 mg, 0.207 mmol) was stirred at r.t. in MeCN (5 mL) for 20 min. The resulting purple solution was concentrated to 1 mL and 10 mL of Et₂O were added. A purple powder precipitated and was isolated by filtration. Yield: 170 mg, 87%. ³¹P{¹H} NMR (121 MHz, CD₃CN): 35.3 ppm.

### (c) Bis(acetonitrile)N¹,N²-bis(2-(dixylylphosphino)benzyl)(R,R)-cyclohexane-1,2-diamine iron(II)tetrafluoroborate, [Fe(NCMe)₂((R,R)-cyPAr₂(NH)₂)][BF₄]₂

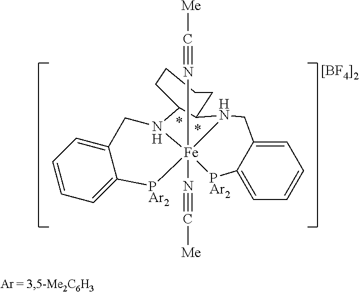

A solution of N¹,N²-bis(2-(dixylylphosphino)benzyl)(R,R)-cyclohexane-1,2-diamine (R,R)-cyPAr₂(NH)₂ (161 mg, 0.207 mmol) and iron(II)tetrafluoroborate hexahydrate [Fe(OH₂)₆][BF₄]₂ (70 mg, 0.207 mmol) was stirred at r.t. in MeCN (5 mL) for 20 min. The resulting purple solution was concentrated to 1 mL and 10 mL of Et₂O were added. A purple powder precipitated and was isolated by filtration. 3: Yield: 190 mg, 91%. ³¹P{¹H} NMR (121 MHz, CD₃CN): 39.2 ppm.

### (d) Bis(acetonitrile)N¹,N²-bis(2-(3,5-di-tert-butyl-4-methoxy-phenylphosphino)benzyl)(R,R)-cyclohexane-1,2-diamine iron(II)tetrafluoroborate, [Fe(NCMe)₂((R,R)-cyPAr₂(NH)₂)][BF₄]₂

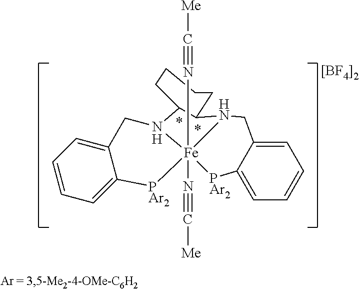

A solution of N¹,N²-bis(2-(3,5-di-tert-butyl-4-methoxy-phenylphosphino)benzyl)(R,R)-cyclohexane-1,2-diamine (R,R)-cyPAr₂(NH)₂ (255 mg, 0.207 mmol) and iron(II)tetrafluoroborate hexahydrate [Fe(OH₂)₆][BF₄]₂ (70 mg, 0.207 mmol) was stirred at r.t. in MeCN (5 mL) for 20 min. The resulting brown solution was concentrated to 1 mL and 10 mL of Et₂O were added. A beige-brown powder precipitated and was isolated by filtration. Yield: 190 mg, 91%. ³¹P{¹H} NMR (121 MHz, CD₃CN): 47.5 ppm.

### (e) Bis(acetonitrile)N¹,N²-bis(2-(diphenylphosphino)benzylidene)(R,R)-diphenylethylene-1,2-diamine iron(II)tetrafluoroborate, [Fe(NCMe)₂((R,R)-dpenPPh₂N₂)][BF₄]₂

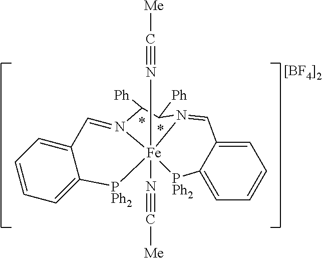

Synthesis of [Fe(NCMe)₂((R,R)-dpenPPh₂N₂)][BF₄]₂, (5). A solution of (1R,2R)-(+)-1,2-diphenylethylenediamine (63 mg, 0.297 mmol), 2-(diphenylphosphino)benzaldehyde (172 mg, 0.593 mmol), and iron(II)tetrafluoroborate hexahydrate [Fe(OH₂)₆][BF₄]₂ (100 mg, 0.296 mmol) was stirred overnight under reflux in MeCN (5 mL). The red orange solution was concentrated to 1 mL and 10 mL of Et₂O were added. A red-orange powder precipitated and was isolated by filtration. Yield: 290 mg, 92%. ³¹P{¹H} NMR (121 MHz, CD₃CN): 52.3 ppm.

### (f) [Fe(CN^{t}Bu)(NCMe)((R,R)-dpenPPh₂N₂)][BF₄]₂

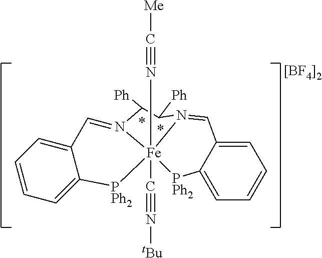

A solution of [Fe(NCMe)₂((R,R)-dpen-PPh₂N₂)][BF₄]₂ (130 mg, 0.122 mmol) and tBuNC (14 μL, 0.122 mmol) in acetone (3 mL) was stirred for 15 min. The resulting orange-yellow solution was evaporated to dryness to give an orange powder). 6: Yield: 55 mg, 41%. ³¹P{¹H} NMR (121 MHz, CD₃CN): 56.1 (²J_{P-P}=48 Hz), 44.8 (²J_{P-P}=48 Hz) ppm.

### (g) [Fe(CMBu)(NCMe)((R,R)-cyPPh₂N₂)][BAr^{F}]₂

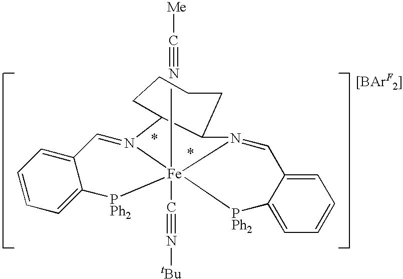

A solution of [Fe(NCMe)₂((R,R)-cy-PPh₂N₂)][BF₄]₂ (40 mg, 0.039 mmol) and NaBAr^{F} (71 mg, 0.079 mmol) in dichloromethane (5 mL) was stirred for 1 hour. The resulting orange-yellow solution was filtered on celite and evaporated to dryness to give an orange powder. 7: Yield: 90 mg, 89%. ³¹P{¹H} NMR (121 MHz, CD₃CN): 55.2 (²J_{P-P}=54 Hz), 48.1 (²J_{P-P}=54 Hz) ppm.

### (h) [Fe(CO)(NCMe)((R,R)-dpenPPh₂N₂)][BF₄]₂

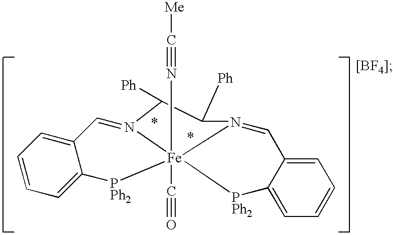

A solution of Fe(NCMe)₂((R,R)-dpenPPPh₂N₂)][BF₄]₂ (185 mg, 0.173 mmol) in acetone (10 mL) was stirred under CO overnight. The resulting orange solution was evaporated to dryness to give an orange powder. The NMR of the crude product shows an AB pattern characteristic of the formation of [Fe(CO)(NCMe)((R,R)-dpenPh₂N₂)][BF₄]₂, (7) (purity<50%) with other unidentified impurities. ³¹P{¹H} NMR (121 MHz, CD₃CN): 52.9 (²J_{P-P}=40 Hz), 49.7 (²J_{P-P}=40 Hz), 9.1, −2.4, −19.6, −22.1 ppm.

### Example 9

### General Procedure for Hydrogenation with Ruthenium Complexes

A solution of acetophenone (1.0 g, 8.3 mmol) in 2-propanol (10 ml) was added to a 50 mL Schlenk flask. After evacuating and refilling with argon, a mixture of catalyst (e.g. [RuCl(R-binap)(R,R-cydn)]BF₄; 0.01 mmol) and K^{t}OBu (20 mg, 0.18 mmol) was added. The resulting mixture was then injected into a 100 mL autoclave which had been previously placed under an atmosphere of H₂. The autoclave was pressurized to 200 psig and the reaction mixture was stirred at ambient temperature. The reaction progress was monitored by TLC. Upon completion of the reaction, the solvent was removed under vacuum and the mixture was filtered through silica gel (ca. 6 cm) using 3:1 hexane:ethyl acetate. The solvent was removed from the filtrate affording the product as a colorless liquid. Results are shown in Tables 1-9.

### Example 10

### Hydrogenation of 2,3,3-trimethylindolenine

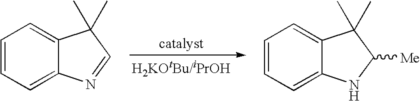

A solution of 2,3,3-trimethylindolenine (0.286 g, 1.8 mmol) in 2-propanol (10 mL) was added to a 50 mL Schlenk flask. After evacuating and refilling with argon, a mixture of catalyst (0.01 mmol) and KO^{t}Bu (29 mg, 0.26 mmol) was added. The resulting mixture was then injected into a 100 mL autoclave which had been previously placed under an atmosphere of H₂. The autoclave was pressurized to 150 psi and the reaction mixture was stirred at ambient temperature. A solution of Na₂CO₃ was added to render the mixture basic. The product was extracted with CH₂Cl₂. The resulting organic phases were dried on MgSO₄, filtered and evaporated to dryness. The ¹H NMR analysis was used to calculate the conversion. The sample was purified by chromatography on silica gel using hexane and ethyl acetate and submitted for HPLC analysis to determine the e.e. The results are presented in Table 10.

### Example 11

### Hydrogenation of Norcamphor

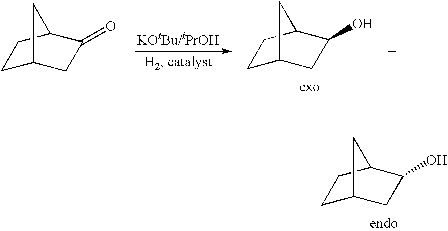

A solution of norcamphor (0.64 g, 5.82 mmol) in 2-propanol (5 mL) was added to a 50 mL Schlenk flask. After evacuating and refilling with argon, a mixture of catalyst (i.e. [RuCl(Ph₂PCH₂CH₂NH₂)₂]BF₄; 0.010 g, 0.015 mmol) and K^{t}OBu (0.02 g, 0.18 mmol) in 2-propanol (5 mL) was added. The resulting mixture was then injected into a 100 mL autoclave which had been previously placed under an atmosphere of H₂. The autoclave was pressurized to 200 psig and the reaction mixture was stirred at ambient temperature. The reaction progress was monitored ¹H NMR. Results for [RuCl(Ph₂PCH₂CH₂NH₂)₂]BF₄:99:1 endo:exo.

### Example 12

### Hydrogenation of Acetophenone Using Cationic Iron Complexes

### (a) H₂ Conditions

Under argon, a solution of degassed acetophenone (120 mg, 1 mmol) and KO^{t}Bu (4.5 mg, 0.04 mmol) was added to a Schlenk flask. The resulting mixture was then injected into a 100 mL autoclave which already contains the iron catalyst (5 mg, 0.005 mmol) and 6 mL of degassed 2-propanol. under an atmosphere of H₂. The autoclave was pressurized to 25 atm and the reaction mixture was stirred at 50° C. After 17 hours, the sample was then filtered through silica gel (ca. 2 cm) using CH₂Cl₂ and submitted for GC analysis. The results are shown in Table 11.

### (b) Transfer Hydrogenation Conditions

Under argon, the iron complex (5 mg, 0.005 mmol), KOtBu (5 mg, 0.045 mmol) and acetophenone (120 mg, 200 equiv) were stirred in 5 mL of 2-propanol at r.t. The sample was then filtered through silica gel (ca. 2 cm) using CH₂Cl₂ and submitted for GC analysis. The results are shown in Table 12.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
RESULTS OF THE HYDROGENATION OF ACETOPHENONE
CATALYSED BY CATIONIC COMPLEXES OF RUTHENIUM

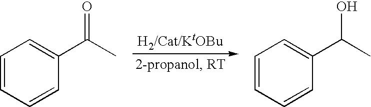

| Entry | Complex | Time (h) | Conv. (%) | ee (%) |
|---|---|---|---|---|
| 1 | [RuCl(R-binap)(R,R-cydn)]BF₄ | 1 | >99 | 80 |
| 2 | [RuCl(R-binap)(R,R-cydn)CH₃CN]BF₄ | 17 | >99 | 84 |
| 3 | [RuCl(R-binap)(R,R-cydn)py]BF₄ | 17 | >99 | 83 |
| 4 | [RuCl(R-binap)(R,R-cydn)][B(C₆F₅)₄] | 17 | >99 | 82 |
| 5 | [RuCl(R-binap)(Ph₂PCH₂CH₂NH₂)]BF₄ | 2 | >99 | 84 |
| 6 | [RuCl(R-binap)(Ph₂PCH₂CH₂NH₂)]B(C₆F₅)₄ | 2 | >99 | 83 |
| 7 | [RuCl(S-binap)(S,S-Ph₂PCH(Ph)CH(Ph) | 17 | >99 | 26 |
| | NH₂)]BF₄ | | | |
| 8 | [RuCl(R-binap)(S,S-Ph₂PCH(Ph)CH(Ph) | 3 | >99 | 69 |
| | NH₂)]BF₄ | | | |
| 9 | [RuCl(Ph₂PCH₂CH₂NH₂)₂]BF₄ | 1 | >99 | n/a |
| 10 | [RuCl(Ph₂PCH₂CH₂NH₂)₂][B(C₆F₅)₄] | 0.5 | >99 | n/a |
| 11 | [RuCl(R-tolbinap)(R,R- | 7 | 70 | 52 |
| | Ph₂PCH(Ph)CH(CH₃)NH₂]BF₄ | | | |
| 12 | [RuCl(R-tolBinap)(R,R- | 8 | >99 | 51 |
| | Ph₂PCH(Ph)CH(CH₃)NH₂][B(C₆F₅)₄] | | | |
| 13 | [RuCl(R-3,5-xylylbinap)(R,R- | 17 | 70 | 20 |
| | Ph₂PCH(Ph)CH(CH₃)NH₂]BF₄ | | | |
| 14 | [RuCl(R-3,5-xylylbinap)(R,R- | 7 | 60 | 33 |
| | Ph₂PCH(Ph)CH(CH₃)NH₂][B(C₆F₅)₄] | | | |
| 15 | [RuCl(PPh₃)₂(NH2CH2—Py)]BF₄ | 3 | >99 | n/a |
| 16 | [RuCl(PPh₃)₂(NH₂CH₂—Py)][B(C₆F₅)₄] | 3 | >99 | n/a |
| 17 | [RuCl(R-Binap)(Sc,Rp-NH₂—CH(CH₃)—Fc— | 22 | 41 | 15 |
| | PPh₂)]BF₄ | | | |
| 18 | [RuCl(R-Binap)(Rc,Sp-NH₂—CH(CH₃)—Fc— | 22 | 85 | 0.5 |
| | PPh₂)]BF₄ | | | |
| 19 | [RuCl(R-Binap)(Sc,Rp-NH₂—CH(CH₃)—Fc— | 22 | 38 | 6.4 |
| | PPh₂)]B(C₆F₅)₄ | | | |
| 20 | [RuCl(R-Binap)(Rc,Sp-NH₂—CH(CH₃)—Fc— | 22 | 63 | 14 |
| | PPh₂)][B(C₆F₅)₄] | | | |
| 21 | [RuCl(1R,2R-(Ph₂PC₆H₄CH₂NH)₂C₆H₁₀)]BF₄ | 1.5 | >99 | 24.3 |
| 22 | [RuCl(1R,2R-(Ph₂PC₆H₄CH₂NH)₂C₆H₁₀)] | 1.5 | >99 | 6.7 |
| | [B(C₆F₅)₄] | | | |
| 23 | [RuCl(1R,2R-(4-methyl- | 1.5 | >99 | 23.5 |
| | Ph₂PC₆H₄CH₂NH)₂C₆H₁₀)]BF₄ | | | |
| 24 | [RuCl(1R,2R-(4-methyl- | 1.5 | >99 | 6.4 |
| | Ph₂PC₆H₄CH₂NH)₂C₆H₁₀)] [B(C₆F₅)₄] | | | |
| 25 | [RuCl(1R,2R-(3,5-dimethyl- | 1.5 | >99 | 73 |
| | Ph₂PC₆H₄CH₂NH)₂C₆H₁₀)]BF₄ | | | |
| 26 | [RuCl(1R,2R-(3,5-dimethyl- | 1.5 | >99 | 74.5 |
| | Ph₂PC₆H₄CH₂NH)₂C₆H₁₀)][B(C₆F₅)₄] | | | |
| 27 | RuCl₂(S-PPhos)(S-DAIPEN) | 5 | >99 | 71 |
| | (Comparative Example) | | | |
| 28 | [RuCl(S-PPhos)(S-DAIPEN)]BF₄ | 4 | >99 | 89.3 |
| 29 | [RuCl(S-PPhos)(S-DAIPEN)]B(C₆F₅)₄ | 4 | >99 | 88.7 |
| 30 | RuCl₂(S-xylylPPhos)(S-DAIPEN) | 3 | >99 | 97.3 |
| | (Comparative Example) | | | |
| 31 | [RuCl(S-xylylPPhos)(S-DAIPEN)]BF₄ | 4 | >99 | 99.3 |
| 32 | [RuCl(S-xylylPPhos)(S-DAIPEN)]B(C₆F₅)₄ | 4 | >99 | 98.7 |
| 33 | RuCl₂(S-BINAP)(S-DAIPEN) | 3 | >99 | 87 |
| | (Comparative Example) | | | |
| 34 | [RuCl(S-BINAP)(S-DAIPEN)]BF₄ | 4 | >99 | 85 |
| 35 | [RuCl(S-BINAP)(S-DAIPEN)]B(C₆F₅)₄ | 4 | >99 | 87 |
| 36 | [RuCl₂(S-PhanePhos)(R,R-DPEN)] | 3 | >99 | 95.6 |
| | (Comparative Example) | | | |
| 37 | [RuCl(S-PhanePhos)(R,R-DPEN)]BF₄ | 3 | >99 | 97.5 |
| 38 | [RuCl(S-PhanePhos)(R,R-DPEN)]B(C₆F₅)₄ | 3 | >99 | 98 |
| 39 | [RuCl₂(S-XylylPhanePhos)(R,R-DPEN)] | 3 | >99 | 96.8 |
| | (Comparative Example) | | | |
| 40 | [RuCl(S-XylylPhanePhos)(R,R-DPEN)]BF₄ | 3 | >99 | 96.6 |
| 41 | [RuCl(S-XylylPhanePhos)(R,R- | 3 | >99 | 97.8 |
| | DPEN)]B(C₆F₅)₄ | | | |
| 42* | [RuCl(PPh₃)₂((S)-1-(pyridin-2- | 1.5 | 65 | 15.5 |
| | yl)ethanamine)]BF₄ | 5 | >99 | 15.5 |
| 43* | [RuCl(S-XylylPPhos)((S)-1-(pyridin-2- | 3 | >99 | 53 |
| | yl)ethanamine)]BF₄ | | | |
| 44* | [RuCl(R-BINAP)((S)-1-(pyridin-2- | 3 | >99 | 3 |
| | yl)ethanamine)]BF₄ | | | |
| 45* | [RuCl(S_{C},Rₚ-PCy₂—CH(CH₃)—Fc—PCy₂)((S)-1- | 1.5 | >99 | 23.7 |
| | (pyridin-2-yl)ethanamine)]BF₄ | | | |
| 46 | [RuCl(R-binap)(R,R-cydn)(dmf)ₙ]BF₄ | 4 | 100 | 82 |
| 47 | [RuCl(R-binap)(R,R-cydn)(dmf)ₙ]B(C₆F₅)₄ | 4 | 100 | 80 |

Substrate: Ru = 830, P_{H2} = 160 Psi.
*Substrate:Ru:Base = 1000:1:12, P_{H2} = 170 psi

TABLE 2
RESULTS OF THE HYDROGENATION OF ACETOPHENONE
CATALYSED BY CATIONIC COMPLEXES OF RUTHENIUM
COMPARING DIFFERENT COUNTER ANIONS.

| | | | Conv. | |
|---|---|---|---|---|
| Entry | Cat | Time (h) | (%) | ee (%) |
| 1 | (R-binap)RuCl₂(R,R-dach) | 17 | >99 | 83 |
| 2 | [(R-binap)RuCl(R,R-dach)]BF₄ | 1 | >99 | 80 |
| 3 | [(R-binap)RuCl(R,R-dach)]PF₆ | 17 | >99 | 78 |
| 4 | [(R-binap)RuCl(R,R-dach)]OTf | 17 | >99 | 78 |
| 5 | [(R-binap)RuCl(R,R-dach)]B(C₆F₅)₄ | 17 | >99 | 82 |
| 6 | [(R-binap)RuCl(R,R-dach)][B(3,5-(CF₃)₂C₆H₃)₄] | 17 | >99 | 82 |
| 7 | (R-binap)RuCl₂(PGly) | 17 | >99 | 23 |
| 8 | [(R-binap)RuCl(PGly)]BF₄ | 2 | >99 | 84 |
| 9 | [(R-binap)RuCl(PGly)]PF₆ | 17 | 99 | 73 |
| 10 | [(R-binap)RuCl(PGly)]OTf | 17 | >99 | 30 |
| 11 | [R-binap)RuCl(PGly)]B(C₆F₅)₄ | 2 | >99 | 83 |
| 12 | [(R-binap)RuCl(PGly)][B(3,5-(CF₃)₂C₆H₃)₄]. | 17 | >99 | 43 |
| 13 | RuCl₂(PGly)₂ | 17 | >99 | — |
| 14 | [RuCl(PGly)₂]BF₄ | 1 | 99 | — |
| 15 | [RuCl(PGly)₂]PF₆ | 17 | >99 | — |
| 16 | [RuCl(PGly)₂]OTf | 17 | >99 | — |
| 17 | [RuCl(PGly)₂]B(C₆F₅)₄ | 0.5 | >99 | — |
| 18 | [RuCl(PGly)₂][B(3,5-(CF₃)₂C₆H₃)₄]. | 17 | >99 | — |
| 19 | [(R-binap)RuCl(R,R-dach)(MeCN)]BF₄ | 17 | >99 | 84 |
| 20 | [(R-binap)RuCl(R,R-dach)(pyr)]BF₄ | 17 | >99 | 83 |

^{a} S:C:B = 830:1:18, iPrOH, r.t., P_{H2} = 150 psi

TABLE 3
RESULTS OF THE HYDROGENATION OF 4-FLUORO-
ACETOPHENONE CATALYSED BY CATIONIC
COMPLEXES OF RUTHENIUM.

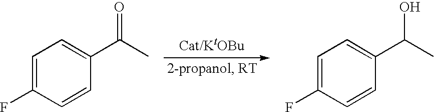

| | | Time | Conv. | ee |
|---|---|---|---|---|
| Entry | Complex | (h) | (%) | (%) |
| 1 | [RuCl(S-PPhos)(S-DAIPEN)]BF₄ | 4 | >99 | 73.6 |
| 2 | [RuCl(S-PPhos)(S- | 4 | >99 | 77 |
| | DAIPEN)]B(C₆F₅)₄ | | | |
| 3 | [RuCl(S-xylylPPhos)(S- | 4 | >99 | 99.1 |
| | DAIPEN)]BF₄ | | | |
| 4 | [RuCl(S-xylylPPhos)(S- | 4 | >99 | 99 |
| | DAIPEN)]B(C₆F₅)₄ | | | |
| 5 | [RuCl(S-BINAP)(S-DAIPEN)]BF₄ | 4 | >99 | 75.4 |
| 6 | [RuCl(S-BINAP)(S- | 4 | >99 | 78.8 |
| | DAIPEN)]B(C₆F₅)₄ | | | |

Substrate: Ru = 830, P_{H2} = 160 Psi

TABLE 4
RESULTS OF THE HYDROGENATION OF 3,5-
BIS(TRIFLUOROMETHYL)-ACETOPHENONE CATALYSED
BY CATIONIC COMPLEXES OF RUTHENIUM.

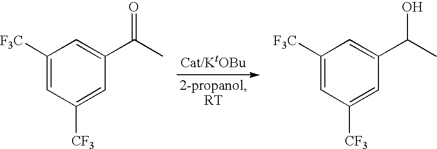

| | | Time | Conv. | ee |
|---|---|---|---|---|
| Entry | Complex | (h) | (%) | (%) |
| 1 | [RuCl(S-PPhos)(S-DAIPEN)]BF₄ | 2 | >99 | 74 |
| 2 | [RuCl(S-PPhos)(S- | 2 | >99 | 75.5 |
| | DAIPEN)]B(C₆F₅)₄ | | | |
| 3 | [RuCl(S-xylylPPhos)(S- | 2 | >99 | 99.0 |
| | DAIPEN)]BF₄ | | | |
| 4 | [RuCl(S-xylylPPhos)(S- | 2 | >99 | 99.0 |
| | DAIPEN)]B(C₆F₅)₄ | | | |
| 5 | [RuCl(S-BINAP)(S-DAIPEN)]BF₄ | 2 | >99 | 77 |
| 6 | [RuCl(S-BINAP)(S- | 2 | >99 | 78.7 |
| | DAIPEN)]B(C₆F₅)₄ | | | |

TABLE 5
RESULTS OF THE HYDROGENATION OF 3-TRIFLUORO-
METHYLACETOPHENONE CATALYSED BY CATIONIC
COMPLEXES OF RUTHENIUM.

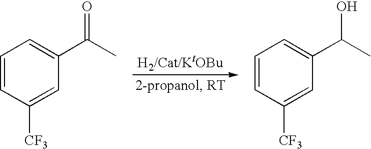

| | | Time | Conv. | ee |
|---|---|---|---|---|
| entry | Cat. | (h) | (%) | (%) |
| 1 | [RuCl(S-PPhos,S-DAIPEN)]BF₄ | 1 | >99 | 76 |
| 2 | [RuCl(S-PPhos,S-DAIPEN)]B(C₆F₅)₄ | 1 | >99 | 81 |
| 3 | [RuCl(S-xylylPPhos,S-DAIPEN)]BF₄ | 1 | >99 | 98.7 |
| 4 | [RuCl(S-xylylPPhos,S-DAIPEN)]B(C₆F₅)₄ | 1 | >99 | 98.5 |
| 5 | [RuCl(S-BINAP,S-DAIPEN)]BF₄ | 1 | >99 | 76.5 |
| 6 | [RuCl(S-BINAP,S-DAIPEN)]B(C₆F₅)₄ | 1 | >99 | 76.7 |
| 7 | [RuCl₂(S)-PhanePhos,(R,R)DPEN] | 17 | >99 | 65.3 |
| 8 | [RuCl(S)-PhanePhos,(R,R)DPEN]BF₄ | 17 | >99 | 78.5 |
| 9 | [RuCl(S)PhanePhos,(R,R)DPEN]B(C₆F₅)₄ | 17 | >99 | 81.3 |
| 10 | [RuCl₂(S)-XylylPhanePhos,(R,R)DPEN] | 17 | >99 | 75.7 |
| 11 | [RuCl(S)-XylylPhanePhos,(R,R)DPEN] | 17 | 97 | 71 |
| | BF₄ | | | |

TABLE 6
RESULTS OF THE HYDROGENATION OF 2-FLUORO-
ACETOPHENONE CATALYSED BY CATIONIC COMPLEXES
OF RUTHENIUM.

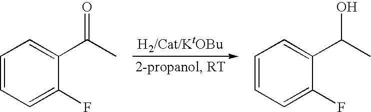

| | | Time | Conv. | ee |
|---|---|---|---|---|
| entry | Cat . | (h) | (%) | (%) |
| 1 | [RuCl(S-PPhos,S-DAIPEN)]BF₄ | 2 | >99 | 80 |
| 2 | [RuCl(S-PPhos,S-DAIPEN)]B(C₆F₅)₄ | 2 | >99 | 80.3 |
| 3 | [RuCl(S-xylylPPhos,S-DAIPEN)]BF₄ | 2 | >99 | 95.3 |
| 4 | [RuCl(S-xylylPPhos,S- | 2 | >99 | 92.4 |
| | DAIPEN)]B(C₆F₅)₄ | | | |
| 5 | [RuCl(S-BINAP,S-DAIPEN)]BF₄ | 2 | >99 | 80.7 |
| 6 | [RuCl(S-BINAP,S-DAIPEN)]B(C₆F₅)₄ | 2 | >99 | 84.6 |
| 7 | [RuCl₂(S)-PhanePhos,(R,R)DPEN] | 3 | 95 | 77.7 |
| 8 | [RuCl(S)-PhanePhos,(R,R)DPEN]BF₄ | 3 | 83 | 80 |
| 9 | [RuCl(S)PhanePhos, | 3 | 98 | 67 |
| | (R,R)DPEN]B(C₆F₅)₄ | | | |
| 10 | [RuCl₂(S)-XylylPhanePhos,(R,R)DPEN] | 3 | 92 | 89.2 |
| 11 | [RuCl(S)-XylylPhanePhos,(R,R)DPEN] | 3 | 63 | 82 |
| | BF₄ | | | |
| 12 | [RuCl(S)-XylylPhanePhos,(R,R)DPEN] | 3 | 93 | 87.3 |
| | B(C₆F₅)₄ | | | |

TABLE 7
RESULTS OF THE HYDROGENATION OF 1-(2,4-
DIMETHOXYPHENYL)ETHANONE CATALYZED BY
RUTHENIUM COMPLEXES OF PHANEPHOS.

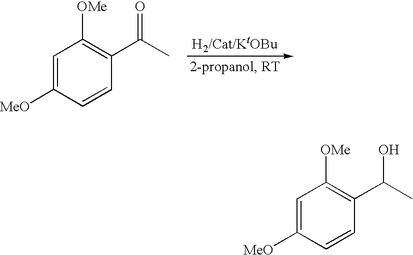

| | | Time | Conv. | ee |
|---|---|---|---|---|
| Entry | Cat. | (h) | (%) | (%) |
| 1 | [RuCl₂(S)-PhanePhos,(R,R)DPEN] | 3 | >99 | 79.3 |
| 2 | [RuCl(S)-PhanePhos,(R,R)DPEN]BF₄ | 3 | >99 | 58.5 |
| 3 | [RuCl(S)PhanePhos,(R,R)DPEN]B(C₆F₅)₄ | 3 | >99 | 75 |
| 4 | [RuCl₂(S)-XylylPhanePhos,(R,R)DPEN] | 3 | >99 | 72 |
| 5 | [RuCl(S)-XylylPhanePhos,(R,R)DPEN]BF₄ | 3 | >99 | 47 |
| 6 | [RuCl(S)-XylylPhanePhos,(R,R)DPEN] | 3 | >99 | 57.7 |
| | B(C₆F₅)₄ | | | |

TABLE 8
RESULTS OF THE HYDROGENATION OF 1-(4-
METHOXYPHENYL)PROPAN-2-ONE CATALYZED BY
RUTHENIUM COMPLEXES OF PHANEPHOS.

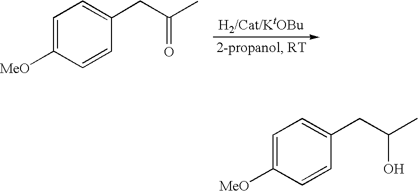

| | | Time | Conv. | ee |
|---|---|---|---|---|
| Entry | Cat. | (h) | (%) | (%) |
| 1 | [RuCl₂(S)-PhanePhos,(R,R)DPEN] | 5 | 99 | 81 |
| 2 | [RuCl(S)-PhanePhos,(R,R)DPEN]BF₄ | 5 | >99 | 79 |
| 3 | [RuCl(S)PhanePhos,(R,R)DPEN]B(C₆F₅)₄ | 5 | >99 | 79 |
| 4 | [RuCl₂(S)-XylylPhanePhos,(R,R)DPEN] | 5 | >99 | 85 |
| 5 | [RuCl(S)-XylylPhanePhos,(R,R)DPEN]BF₄ | 5 | >88 | 75 |
| 6 | [RuCl(S)-XylylPhanePhos,(R,R)DPEN] | 5 | >99 | 78 |
| | B(C₆F₅)₄ | | | |

TABLE 9
RESULTS OF THE HYDROGENATION OF ACETOPHENONE
CATALYSED BY DICATIONIC COMPLEXES OF RUTHENIUM

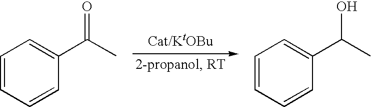

| | | Time | Conv. | ee |
|---|---|---|---|---|
| Entry | Complex | (h) | (%) | (%) |
| 1 | [Ru(R-binap)(R,R-cydn)][BF₄]₂ | 2 | >99 | 85 |
| 2 | [Ru(R-binap)(Ph₂PCH₂CH₂NH₂)][BF₄]₂ | 24 | 93 | 3 |
| 3 | [Ru(1R,2R-(Ph₂PC₆H₄CH₂NH)₂C₆H₁₀)][BF₄]₂ | 24 | 94 | 10 |

S/Cat = 830

TABLE 10
RESULTS OF THE HYDROGENATION OF 2,3,3-
TRIMETHYLINDOLENINE CATALYZED BY
CATIONIC RUTHENIUM COMPLEXES.

| | | | Conv. | |
|---|---|---|---|---|
| Entry | Cat | Time (h) | (%) | e.e. (%) |
| 1 | [(R-binap)RuCl(R,R-dach)]BF₄ | 17 | 57 | 30 |
| 2 | [(R-binap)RuCl(PGly)]BF₄ | 17 | 5 | 40 |
| 3 | [RuCl(PGly)₂]BF₄ | 17 | 62 | — |
| 4 | [RuCl(R,R-cyP₂N₂)]BF₄ | 17 | 16 | 76 |

^{a} S:C:B = 180:1:26, iPrOH, r.t., 150 psi

TABLE 11
CATALYTIC HYDROGENATION OF ACETOPHENONE USING
CATIONIC IRON COMPLEXES^{A}

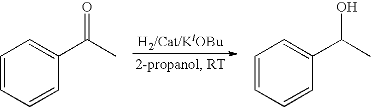

| | Catalyst C | | | | | Conv. | ee |
|---|---|---|---|---|---|---|---|
| Entry | (Ex. #) | S:C:B | T (° C.) | P_{H2} (atm) | Time (h) | (%) | (%) |
| 1 | 6(a) | 225:1:15 | 50 | 25 | 17 | 7 | 1 |
| 2 | 6(b) | 225:1:15 | 50 | 25 | 17 | 3 | 8 |
| 3 | 6(c) | 225:1:15 | 50 | 25 | 17 | 4 | 20 |
| 4 | 6(d) | 200:1:8 | 50 | 25 | 17 | 4 | 20 |
| 5 | 6(e) | 200:1:8 | 50 | 25 | 17 | 0 | — |
| 6 | 6(f) | 200:1:8 | 50 | 25 | 17 | 3 | — |
| 7 | 6(h) | 200:1:8 | 50 | 25 | 17 | 69 | 58 |
| 8 | 6(h) | 200:1:8 | 50 | 25 | 17 | 96 | 63 |
| 9 | 6(h) | 200:1:1 | 50 | 25 | 17 | 95 | 53 |

^{A}S:C:B = 200:1:8, S = PhCOMe, C = catalyst, B = KO^{t}Bu;
[S] = 0.36 M in 5 mL of i-PrOH.

TABLE 12
TRANSFER HYDROGENATION OF ACETOPHENONE USING
CATIONIC IRON COMPLEXES^{A}

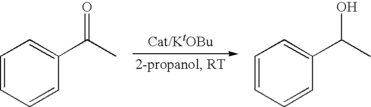

| | Catalyst C | | | | Conv. | ee |
|---|---|---|---|---|---|---|
| Entry | (Example #) | S:C:B | T (° C.) | Time (h) | (%) | (%) |
| 1 | 6(a) | 200:1:10 | 25 | 19 | 0 | — |
| 2 | 6(a) | 200:1:10 | 80 | 19 | 0 | — |
| 3 | 6(e) | 200:1:10 | 80 | 19 | 0 | — |
| 4 | 6(f) | 200:1:10 | 25 | 19 | 0 | — |
| 5 | 6(f) | 200:1:10 | 80 | 18 | 19 | 22 |
| 6 | 6(g) | 200:1:10 | 25 | 3 | 12 | 78 |
| 7 | 6(h) | 200:1:10 | 25 | 3 | 6 | 60 |
| 8 | 6(h) | 200:1:10 | 80 | 3 | 3 | — |

^{A}S = PhCOMe, C = catalyst, B = KO^{t}Bu;
[S] = 0.36 M in 5 mL of i-PrOH.

1. A compound of the Formula I:
[Ru(P₂)(PN)X_{q}(LB)ₙ]^{r+}[Y⁻]ᵣ (I)
wherein
P₂ is a bidentate bisphosphino ligand of the Formula (VII):
R⁴R⁵P-Q¹-PR⁶R⁷ (VII)
wherein
R⁴, R⁵, R⁶ and R⁷ are independently selected from C₆₋₁₈aryl, C₁₋₂₀alkyl and C₃₋₂₀cycloalkyl, each being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl;
Q¹ is selected from unsubstituted or substituted C₁-C₁₀alkylene and unsubstituted or substituted C₁-C₈alkenylene where the substituents on Q¹ are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl, and/or
two substituents on Q¹ are joined together to form, including the carbon atoms to which they are attached, one or more unsubstituted or substituted 5-20-membered monocyclic, polycyclic, heterocyclic, carbocyclic, saturated, unsaturated or metallocenyl ring systems, where the term substituted with respect to the Q¹ substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl;
Q¹ is chiral or achiral;
PN is a ligand of the Formula (VIII):
R⁸R⁹P-Q²-NR¹⁰R¹¹ (VIII)
wherein R⁸ and R⁹ are independently selected from C₆₋₁₈aryl, C₁₋₂₀alkyl and C₃₋₂₀cycloalkyl, each being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl;
Q² is selected from unsubstituted or substituted C₁-C₁₀alkylene and unsubstituted or substituted C₁-C₈alkenylene where the substituents on Q² are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl, and/or two substituents on Q² are joined together to form, including the carbon atoms to which they are attached, one or more unsubstituted or substituted 5-20-membered monocyclic, polycyclic, heterocyclic, carbocyclic, saturated, unsaturated or metallocenyl ring systems, where the term substituted with respect to the Q² substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl;
Q² is chiral or achiral;
R¹⁰ and R¹¹ are independently selected from H, C₆₋₁₈aryl, C₁₋₂₀alkyl and C₃₋₁₀cycloalkyl, the latter three groups being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl, wherein at least one of R¹⁰ and R¹¹ is H;
X is any anionic ligand;
LB is any neutral Lewis base;
Y is any non-coordinating anion;
n is 0, 1 or 2;
q is 0 or 1;
r is 1 or 2; and
q+r=2.

2. The compound according to claim 1, wherein R⁴, R⁵, R⁶ and R⁷ are independently selected from phenyl, C₁₋₆alkyl and C₃₋₁₀cycloalkyl, each being optionally substituted with one to three substituents independently selected from C₁₋₄alkyl, fluoro-substituted C₁₋₄alkyl, halo, C₁₋₄alkoxy and fluoro-substituted C₁₋₄alkoxy;
Q¹ is selected from unsubstituted or substituted C₁-C₈alkylene where the substituents on Q¹ are independently selected from one to four C₁₋₄alkyl, fluoro-substituted C₁₋₄alkyl, halo, C₁₋₄alkoxy, fluoro-substituted C₁₋₄alkoxy, unsubstituted and substituted phenyl and substituted and unsubstituted naphthyl, or two substituents are joined together to form, including the carbon atoms to which they are attached, one or more unsubstituted or substituted phenylene, cyclohexylene, naphthylene, pyridylene or ferrocenylene groups; and
Q¹ is chiral or achiral.

3. The compound according to claim 2, wherein R⁴, R⁵, R⁶ and R⁷ are all cyclohexyl, phenyl, xylyl or tolyl.

4. The compound according to claim 1, wherein the bis(phosphino)ligand of the Formula (VII) is selected from:
2,2′-bis-(diphenylphosphino)-1,1′-binaphthyl (BINAP);
2,2′-bis(diphenylphosphino)-5,5′,6,6′,7,7′,8,8′-octahydro-1,1′-binaphthyl (H₈BINAP);
2,2′-bis-(diphenylphosphino)-6,6′-dimethyl-1,1′-binaphthyl (6MeBINAP);
2,2′-bis-(di-p-tolylphosphino)-1,1′-binaphthyl (Tol-BINAP);
2,2′-bis[bis(3-methylphenyl)phosphino]-1,1′-binaphthyl;
2,2′-bis[bis(3,5-di-tert-butylphenyl)phosphino]-1,1′-binaphthyl;
2,2′-bis[bis(4-tert-butylphenyl)phosphino]-1,1′-binaphthyl;
2,2′-bis[bis(3,5-dimethylphenyl)phosphino]-1,1′-binaphthyl (Xyl-BINAP);
2,2′-bis[bis(3,5-dimethyl-4-methoxyphenyl)phosphino]-1,1′-binaphthyl (Dmanyl-BINAP);
2,2′-bis[bis-(3,5-dimethylphenyl)phosphino]-6,6′-dimethyl-1,1′-binaphthyl (Xyl-6MeBINAP);
3,3′-bis-(diphenylphosphanyl)-13,13′-dimethyl-12,13,14,15,16,17,12′,13′,14′,15′,16′,17′-dodecahydro-11H,11′H-[4,4′]bi[cyclopenta[a]phenanthrenyl]; 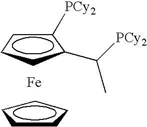
wherein Cy is C₅₋₈cycloalkyl; 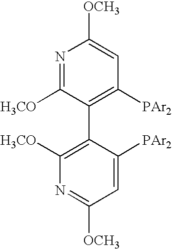
where Ar is phenyl(PPhos), xylyl(XylPPhos) or tolyl(TolPPhos); 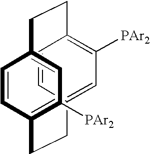
where Ar is phenyl(PhanePhos), xylyl(XylPhanePhos) or tolyl(TolPhanePhos); and
optical isomers thereof and mixtures of optical isomers in any ratio.

5. The compound according to claim 1, wherein R⁸ and R⁹ are independently selected from phenyl, C₁₋₈alkyl and fluoro-substituted C₁₋₈alkyl, with the phenyl being optionally substituted with one to five substituents independently selected from C₁₋₄alkyl, fluoro-substituted C₁₋₄alkyl, halo, C₁₋₄alkoxy and fluoro-substituted C₁₋₄alkoxy;
Q² is selected from unsubstituted or substituted C₁-C₈alkenylene where the substituents on Q² are independently selected from one to four of C₁₋₆alkyl, fluoro-substituted C₁₋₆alkyl, halo, C₁₋₆alkoxy, fluoro-substituted C₁₋₆alkoxy and unsubstituted or substituted phenyl; and/or
adjacent substituents on Q² are joined together to form, including the carbon atoms to which they are attached, one or more unsubstituted or substituted phenylene, naphthylene or ferrocenylene ring systems; or
the term substituted with respect to the Q² substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl; and
Q² is chiral or achiral.

6. The compound according to claim 5, wherein R⁸ and R⁹ are both phenyl, tolyl or xylyl.

7. The compound according to claim 1, wherein R¹⁰ and R¹¹ are both H.

8. The compound according to claim 1, wherein PN is selected from:
Ph₂PCH₂CH₂NH₂ (PGly); and 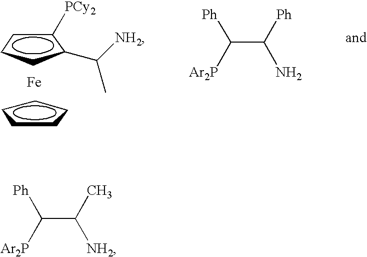
wherein Ar is selected from Ph, tolyl and xylyl; and

9. A compound of the Formula III:
[Ru(P)ₘ(N₂)X_{q}(LB)ₙ]^{r+}[Y⁻]ᵣ (III)
wherein
P is a monodentate phosphine ligand of the Formula (VI):
PR¹R²R³ (VI)
wherein
R¹, R² and R³ are independently selected from C₆₋₁₈aryl, C₁₋₂₀alkyl and C₃₋₂₀cycloalkyl, each being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl,
N₂ is a bidentate diamine ligand of the Formula (X):
R¹⁸R¹⁹N-Q⁶-NR²⁰R²¹ (X)
R¹⁸ and R¹⁹ are independently selected from H, C₆₋₁₈aryl, C₁₋₂₀alkyl and C₃₋₁₀cycloalkyl, the latter three groups being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl, and at least one of R¹⁸ and R¹⁹ is H,
Q⁶ is selected from unsubstituted or substituted C₁-C₁₀alkylene and unsubstituted or substituted C₁-C₈alkenylene where the substituents on Q⁶ are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl, and/or two substituents on Q⁶ are joined together to form, including the carbon atoms to which they are attached, one or more unsubstituted or substituted 5-20-membered monocyclic, polycyclic, heterocyclic, carbocyclic, saturated, unsaturated or metallocenyl ring systems, where the term substituted with respect to the Q⁶ substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl;
Q⁶ is chiral or achiral;
R²⁰ and R²¹ are independently selected from H, C₆₋₁₈aryl, C₁₋₂₀alkyl and C₃₋₁₀cycloalkyl, the latter three groups being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl, and at least of R²⁰ and R²¹ is H, or one of R²⁰ and R²¹ is joined with a substituent on Q⁶ to form, together with the nitrogen atom to which R²⁰ and R²¹ is attached, a pyridine ring and the other of R²⁰ and R²¹ is non-existent;
X is any anionic ligand;
LB is any neutral Lewis base;
Y is any non-coordinating anion;
n is 0, 1 or 2;
m is 1 or 2;
q is 0 or 1;
r is 1 or 2; and
q+r=2.

10. The compound according to claim 9, wherein R¹, R² and R³ are independently selected from phenyl, C₁₋₆alkyl and C₃₋₁₀cycloalkyl, each being optionally substituted with one to three substituents independently selected from C₁₋₆alkyl, fluoro-substituted C₁₋₄alkyl, halo, C₁₋₄alkoxy and fluoro-substituted C₁₋₆alkoxy.

11. The compound according to claim 10, wherein R¹, R² and R³ are all cyclohexyl, phenyl, xylyl or tolyl.

12. The compound according to claim 9, wherein R¹⁸ and R¹⁹ are both H.

13. The compound according to claim 9, wherein R¹⁸, R¹⁹, R²⁰ and R²¹ are all H.

14. The compound according to claim 9, wherein Q⁶ is selected from unsubstituted or substituted C₁-C₈alkenylene where the substituents on Q³ are independently selected from one to four of C₁₋₆alkyl, fluoro-substituted C₁₋₆alkyl, halo, C₁₋₆alkoxy, fluoro-substituted C₁₋₆alkoxy and unsubstituted or substituted phenyl; and/or two substituents on Q⁶ are joined together to form, including the carbon atoms to which they are attached, one or more unsubstituted or substituted phenyl, naphthyl or ferrocenyl ring systems; and
Q⁶ is chiral or achiral.

15. The compound according to claim 9, wherein one of R¹⁸ or R¹⁹ is joined with a substituent on Q⁶ to form, together with the nitrogen atom to which R¹⁸ or R¹⁹ is attached, a pyridine ring and the other of one of R¹⁸ or R¹⁹ is not present.

16. The compound according to claim 9, wherein the compound of the Formula (X) is selected from:
ethylenediamine;
1,2-diaminopropane;
1,3-diaminopropane;
1,4-diaminopropane;
2,3-diaminobutane;
1,2-cyclopentanediamine;
1,2-cyclohexanediamine;
1,2-diphenylethylenediamine (DPEN);
1,1-di(p-methoxyphenyl)ethylenediamine;
1,1-di(3,5-dimethoxyphenyl)ethylenediamine;
1,1-dinaphthylethylenediamine;
1,2-cycloheptanediamine;
2,3-dimethylbutanediamine;
1-methyl-2,2-diphenylethylenediamine (DACH or CYDN);
1-isobutyl-2,2-diphenylethylenediamine;
1-isopropyl-2,2-diphenylethylenediamine;
1-benzyl-2,2-diphenylethylenediamine;
1-methyl-2,2-di(p-methoxyphenyl)ethylenediamine (DAMEN);
1-isobutyl-2,2-di(p-methoxyphenyl)-ethylenediamine (DAIBEN);
1-isopropyl-2,2-di(p-methoxyphenyl)ethylenediamine (DAIPEN);
1-benzyl-2,2-di(p-methoxyphenyl)ethylenediamine;
1-methyl-2,2-di(3,5-dimethoxyphenyl)ethylenediamine;
1-isopropyl-2,2-di(3,5-dimethoxyphenyl)ethylenediamine,
1-isobutyl-2,2-di(3,5-dimethoxy-phenyl)ethylenediamine;
1-benzyl-2,2-di(3,5-dimethoxyphenyl)ethylenediamine;
1-methyl-2,2-dinaphthylethylenediamine;
1-isobutyl-2,2-dinaphthylethylene-diamine;
1-isopropyl-2,2-dinaphthylethylenediamine;
1-benzyl-2,2-dinaphthylethylenediamine; 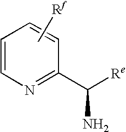
wherein R^{e} is H, C₁₋₆alkyl, fluoro-substituted C₁₋₆alkyl or aryl and R^{f} is H, halo, C₁₋₆alkyl, fluoro-substituted-C₁₋₆alkyl, C₂₋₆alkenyl, C₂₋₆alkynyl, C₃₋₇cycloalkyl, C₁₋₆alkoxy, fluoro-substituted-C₁₋₆alkoxy or C₆₋₁₄aryl; and
optical isomers thereof and mixtures of optical isomers in any ratio.

17. A compound of the Formula (V)
[Ru(P₂)(N₂)X_{q}(LB)ₙ]^{r+}[Y⁻]ᵣ (V)
wherein
P₂ is a bidentate bisphosphino ligand of the Formula (VII):
R⁴R⁵P-Q¹-PR⁶R⁷ (VII)
wherein
R⁴, R⁵, R⁶ and R⁷ are independently selected from C₆₋₁₈aryl, C₁₋₂₀alkyl and C₃₋₂₀cycloalkyl, each being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl;
Q¹ is selected from unsubstituted or substituted C₁-C₁₀alkylene and unsubstituted or substituted C₁-C₈alkenylene where the substituents on Q¹ are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl, and/or
two substituents on Q¹ are joined together to form, including the carbon atoms to which they are attached, one or more unsubstituted or substituted 5-20-membered monocyclic, polycyclic, heterocyclic, carbocyclic, saturated, unsaturated or metallocenyl ring systems, where the term substituted with respect to the Q¹ substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl;
Q¹ is chiral or achiral;
N₂ is a bidentate diamine ligand of the Formula (X):
R¹⁸R¹⁹N-Q⁶-NR²⁰R²¹ (X)
R¹⁸ and R¹⁹ are independently selected from H, C₆₋₁₈aryl, C₁₋₂₀alkyl and C₃₋₁₀cycloalkyl, the latter three groups being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl, and at least one of R¹⁸ and R¹⁹ is H;
Q⁶ is selected from unsubstituted or substituted C₁-C₁₀alkylene and unsubstituted or substituted C₁-C₈alkenylene where the substituents on Q⁶ are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl, and/or
two substituents on Q⁶ are joined together to form, including the carbon atoms to which they are attached, one or more unsubstituted or substituted 5-20-membered monocyclic, polycyclic, heterocyclic, carbocyclic, saturated, unsaturated or metallocenyl ring systems, where the term substituted with respect to the Q⁶ substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl;
Q⁶ is chiral or achiral;
R²⁰ and R²¹ are independently selected from H, C₆₋₁₈aryl, C₁₋₂₀alkyl and C₃₋₁₀cycloalkyl, the latter three groups being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl and at least one of R²⁰ and R²¹ is H, or one of R²⁰ and R²¹ are joined with a substituent on Q⁶ to form, together with the nitrogen atom to which R²⁰ and R²¹ is attached, a pyridine ring and the other of R²⁰ and R²¹ is non-existent;
X is any anionic ligand;
LB is any neutral Lewis base which is coordinated to Ru through a single atom;
Y is any non-coordinating anion;
n is 0, 1 or 2;
q is 0 or 1;
r is 1 or 2; and
q+r=2.

18. The compound according to claim 17, wherein R⁴, R⁵, R⁶ and R⁷ are independently selected from phenyl, C₁₋₆alkyl and C₃₋₁₀cycloalkyl, each being optionally substituted with one to three substituents independently selected from C₁₋₄alkyl, fluoro-substituted C₁₋₄alkyl, halo, C₁₋₄alkoxy and fluoro-substituted C₁₋₄alkoxy;
Q¹ is selected from unsubstituted or substituted C₁-C₈alkylene where the substituents on Q¹ are independently selected from one to four C₁₋₄alkyl, fluoro-substituted halo, C₁₋₄alkoxy, fluoro-substituted C₁₋₄alkoxy, unsubstituted and substituted phenyl and substituted and unsubstituted naphthyl, or two substituents are joined together to form, including the carbon atoms to which they are attached, one or more unsubstituted or substituted phenylene, cyclohexylene, naphthylene, pyridylene or ferrocenylene groups; and
Q¹ is chiral or achiral.

19. The compound according to claim 18, wherein R⁴, R⁵, R⁶ and R⁷ are all cyclohexyl, phenyl, xylyl or tolyl.

20. The compound according to claim 18, wherein the bis(phosphino)ligand of the Formula (VII) is selected from:
2,2′-bis-(diphenylphosphino)-1,1′-binaphthyl (BINAP);
2,2′-bis(diphenylphosphino)-5,5′,6,6′,7,7′,8,8′-octahydro-1,1′-binaphthyl (H₈BINAP);
2,2′-bis-(diphenylphosphino)-6,6′-dimethyl-1,1′-binaphthyl (6MeBINAP);
2,2′-bis-(di-p-tolylphosphino)-1-,1′-binaphthyl (Tol-BINAP);
2,2′-bis[bis(3-methylphenyl)phosphino]-1,1′-binaphthyl;
2,2′-bis[bis(3,5-di-tert-butylphenyl)phosphino]-1,1′-binaphthyl;
2,2′-bis[bis(4-tert-butylphenyl)phosphino]-1,1′-binaphthyl;
2,2′-bis[bis(3,5-dimethylphenyl)phosphino]-1,1′-binaphthyl (Xyl-BINAP);
2,2′-bis[bis(3,5-dimethyl-4-methoxyphenyl)phosphino]-1,1′-binaphthyl (Dmanyl-BINAP);
2,2′-bis[bis-(3,5-dimethylphenyl)phosphino]-6,6′-dimethyl-1,1′-binaphthyl (Xyl-6MeBINAP);
3,3′-bis-(diphenylphosphanyl)-13,13′-dimethyl-12,13,14,15,16,17,12′,13′,14′,15′,16′,17′-dodecahydro-11H,11′H-[4,4′]bi[cyclopenta[a]phenanthrenyl]; 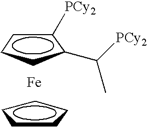
wherein Cy is C₅₋₈cycloalkyl; 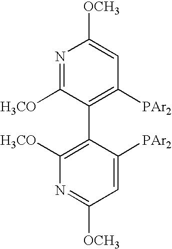
where Ar is phenyl (PPhos), xylyl (XylPPhos) or tolyl (TolPPhos); 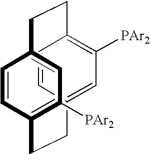
where Ar is phenyl (PhanePhos), xylyl (XylPhanePhos) or tolyl (TolPhanePhos); and
optical isomers thereof and mixtures of optical isomers in any ratio.

21. The compound according to claim 17, wherein Q⁶ is selected from unsubstituted or substituted C₁-C₈alkenylene where the substituents on Q⁶ are independently selected from one to four of C₁₋₆alkyl, fluoro-substituted C₁₋₆alkyl, halo, C₁₋₆alkoxy, fluoro-substituted C₁₋₆alkoxy and unsubstituted or substituted phenyl; and/or
two substituents on Q⁶ are joined together to form, including the carbon atoms to which they are attached, one or more unsubstituted or substituted phenyl, naphthyl or ferrocenyl ring systems; and
Q⁶ is chiral or achiral.

22. The compound according to claim 17, wherein R¹⁸, R¹⁹, R²⁰ and R²¹ are all H.

23. The compound according to claim 17, wherein R²⁰ or R²¹ are joined with a substituent on Q⁶ to form, together with the nitrogen atom to which R²⁰ or R²¹ is attached, a pyridine ring and the other of one of R²⁰ or R²¹ is not present.

24. The compound according to claim 17, wherein N₂ is selected from:
1,2-diaminopropane;
1,3-diaminopropane;
1,4-diaminopropane;
2,3-diaminobutane;
1,2-cyclopentanediamine;
1,2-cyclohexanediamine;
1,1-diphenylethylenediamine (DPEN);
1,1-di(p-methoxyphenyl)ethylenediamine;
1,1-di(3,5-dimethoxyphenyl)ethylenediamine;
1,1-dinaphthylethylenediamine;
1,2-cycloheptanediamine;
2,3-dimethylbutanediamine;
1-methyl-2,2-diphenylethylenediamine (DACH or CYDN);
1-isobutyl-2,2-diphenylethylenediamine;
1-isopropyl-2,2-diphenylethylenediamine;
1-benzyl-2,2-diphenylethylen-ediamine;
1-methyl-2,2-di(p-methoxyphenyl)ethylenediamine (DAMEN);
1-isobutyl-2,2-di(p-methoxyphenyl)-ethylenediamine (DAIBEN);
1-isopropyl-2,2-di(p-methoxyphenyl)ethylenediamine (DAIPEN);
1-benzyl-2,2-di(p-methoxyphenyl)ethylenediamine;
1-methyl-2,2-di(3,5-dimethoxyphenyl)ethylenediamine;
1-isopropyl-2,2-di(3,5-dimethoxyphenyl)ethylenediamine,
1-isobutyl-2,2-di(3,5-dimethoxy-phenyl)ethylenediamine;
1-benzyl-2,2-di(3,5-dimethoxyphenyl)ethylenediamine;
1-methyl-2,2-dinaphthylethylenediamine;
1-isobutyl-2,2-dinaphthylethylene-diamine;
1-isopropyl-2,2-dinaphthylethylenediamine;
1-benzyl-2,2-dinaphthylethylenediamine; 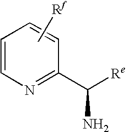
wherein R^{e} is H, C₁₋₆alkyl, fluoro-substituted C₁₋₆alkyl or aryl and R^{f} is H, halo, C₁₋₆alkyl, fluoro-substituted-C₁₋₆alkyl, C₂₋₆alkenyl, C₂₋₆alkynyl, C₃₋₇cycloalkyl, C₁₋₆alkoxy, fluoro-substituted-C₁₋₆alkoxy or C₆₋₁₄aryl; and
optical isomers thereof and mixtures of optical isomers in any ratio.

25. The compound according to claim 1, wherein X is selected from halo, C₁₋₆alkoxy, carboxylate, sulfonates and nitrates.

26. The compound according to claim 1, wherein LB is selected from acetonitrile, DMF and pyridine.

27. The compound according to claim 1, wherein Y is selected from:
a) OTf,
b) BF₄,
c) PF₆,
d) B(C₁₋₆alkyl)₄,
e) B(fluoro-substituted-C₁₋₆alkyl)₄,
f) B(C₆₋₁₈aryl)₄, wherein aryl is unsubstituted or substituted 1-5 times with fluoro, C₁₋₄alkyl or fluoro-substituted C₁₋₄alkyl,
g) 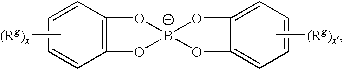
R^{g} is independently halo, C₁₋₄alkyl or fluoro-substituted-C₁₋₄alkyl and x and x′ are independently an integer between 1 and 4,
h) 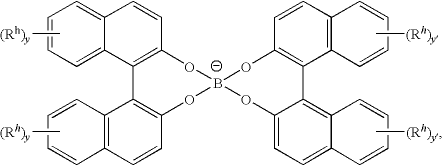
wherein R^{h} is independently halo, C₁₋₄alkyl or fluoro-substituted-C₁₋₄alkyl and y and y′ are independently an integer between 1 and 6,
i) Al(C₁₋₆alkyl)₄,
j) Al(fluoro-substituted-C₁₋₆alkyl)₄,
k) Al(C₆₋₁₈aryl)₄, wherein aryl is unsubstituted or substituted 1-5 times with fluoro, C₁₋₄alkyl or fluoro-substituted
l) Al(—O—C₁₋₆alkyl)₄,
m) Al(—O-fluoro-substituted-C₁₋₆alkyl)₄
n) Al(—O—C₆₋₁₈aryl)₄, wherein aryl is unsubstituted or substituted 1-5 times with fluoro, C₁₋₄alkyl or fluoro-substituted C₁₋₄alkyl
o) a carborane,
p) a bromocarborane; and
q) a phosphate.

28. The compound according to claim 27, wherein the phosphate anion is of the formula 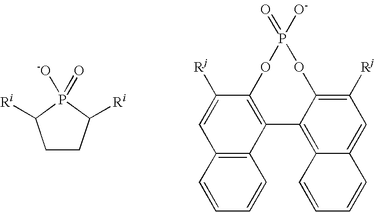
wherein Rⁱ and R^{j} are independently selected from halo, C₁₋₄alkyl, fluoro-substituted-C₁₋₄alkyl or C₆₋₁₈aryl.

29. The compound according to claim 27, wherein the carborane is CB₁₁H₁₂.

30. The compound according to claim 27, wherein the bromocarborane is CB₁₁H₆Br₆.

31. The compound according to claim 27, wherein Y is chiral.

32. A process for preparing a compound of claim 1, comprising combining a compound of the formula
Ru(P₂)(PN)X₂ (XI)
wherein P₂, PN and X are as defined in claim 1, with one or two molar equivalents of an anion abstracting agent and optionally a non- or weakly-coordinating Lewis Base, and reacting under conditions to form the compound and optionally isolating the compound.

33. A process for preparing a compound of claim 1, comprising combining a precursor ruthenium compound with one or two molar equivalents of an anion abstracting agent, and optionally a Lewis Base and reacting under conditions to form a cationic or dicationic precursor ruthenium compound and combining the cationic or dicationic precursor ruthenium compound with one or more P₂, or PN, or ligands, as defined in claim 1, and optionally a non- or weakly-coordinating Lewis Base, under conditions to form the compound and optionally isolating the compound.

34. The process according to claim 33, wherein the precursor ruthenium compound is of the formula [RuX₂(p-ligand)]₂ or RuX₂(ligand), wherein X is as defined in claim 1 and ligand is any displaceable ligand.

35. The process according to claim 34, wherein the displaceable ligand is p-cymene, benzene, cyclooctadiene (COD) or norbornadiene (NBD).

36. The process according to claim 35, wherein the displaceable ligand is p-cymene or NBD.

37. The process according to claim 34, wherein the precursor metal compound is of the formula RuX₂(P₂)(LB)ₙ, wherein X, P₂ and LB are as defined in claim 1 and n is 1 or 2.

38. The process according to claim 37, wherein (P₂) is BINAP and LB is DMF or pyridine.

39. The process according to claim 32, wherein the anion abstracting agent is a salt of a non-coordinating counter anion Y, wherein Y is as defined in claim 27.

40. A method for catalyzing a synthetic organic reaction comprising combining starting materials for the reaction with a compound according to claim 1 under conditions for performing the reaction.

41. The method according to claim 40, wherein the synthetic organic reaction is selected from hydrogenation, transfer hydrogenation, hydroformylation, hydrosilylation, hydroboration, hydroamination, hydrovinylation, hydroarylation, hydration, oxidation, epoxidation, reduction, C—C and C—X bond formation, functional group interconversion, kinetic resolution, dynamic kinetic resolution, cycloaddition, Diels-Alder, retro-Diels-Alder, sigmatropic rearrangement, electrocyclic reactions, ring-opening and/or ring-closing olefin metathesis, carbonylation and aziridination.

42. The method according to claim 41, wherein the C—C and C—X bond formation reaction is selected from Heck, Suzuki-Miyaura, Negishi, Buchwald-Hartwig Amination, α-Ketone Arylation, N-Aryl Amination, Murahashi, Kumada and Stille reactions.

43. The method according to claim 41, wherein the reaction is hydrogenation or transfer hydrogenation

44. The method according to claim 40 wherein the reaction is regioselective, chemoselective, stereoselective or diastereoselective.

45. A method for catalyzing a synthetic organic reaction comprising combining starting materials for the reaction with a compound of the Formula (II):
[Ru(PN)₂X_{q}(LB)ₙ]^{r+}[Y⁻]ᵣ (II)
wherein
PN is a ligand of the Formula (VIII):
R⁸R⁹P-Q²-NR¹⁰R¹¹ (VIII)
wherein R⁸ and R⁹ are independently selected from C₆₋₁₈aryl, C₁₋₂₀alkyl and C₃₋₂₀cycloalkyl, each being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl;
Q² is selected from unsubstituted or substituted C₁-C₁₀alkylene and unsubstituted or substituted C₁-C₈alkenylene where the substituents on Q² are independently selected from one or more of C₁₋₆alkyl, fluoro-substituted C₁₋₆alkyl, halo, C₁₋₆alkoxy, fluoro-substituted C₁₋₆alkoxy and unsubstituted or substituted C₆₋₁₄aryl, and/or
two substituents on Q² are joined together to form, including the carbon atoms to which they are attached, one or more unsubstituted or substituted 5-20-membered monocyclic, polycyclic, heterocyclic, carbocyclic, saturated, unsaturated or metallocenyl ring systems, where the term substituted with respect to the Q² substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl;
Q² is chiral or achiral;
R¹⁰ and R¹¹ are independently selected from H, C₆₋₁₈aryl, C₁₋₂₀alkyl and C₃₋₁₀cycloalkyl, the three groups being optionally substituted with one to five substituents independently selected from C₁₋₆alkyl, fluoro-substituted C₁₋₆alkyl, halo, C₁₋₆alkoxy, fluoro-substituted C₁₋₆alkoxy and C₆₋₁₄aryl, wherein at least one of R¹⁰ and R¹¹ is H;
X is any anionic ligand;
LB is any neutral Lewis base;
Y is any non-coordinating anion;
n is 0, 1 or 2;
q is 0 or 1;
r is 1 or 2; and
q+r=2,
under conditions for performing the reaction.

46. The method according to claim 45, wherein R⁸ and R⁹ are independently selected from phenyl, C₁₋₆alkyl and fluoro-substituted C₁₋₆alkyl, with the phenyl being optionally substituted with one to five substituents independently selected from C₁₋₄alkyl, fluoro-substituted C₁₋₄alkyl, halo, C₁₋₄alkoxy and fluoro-substituted C₁₋₄alkoxy;
Q² is selected from unsubstituted or substituted C₁-C₈alkenylene where the substituents on Q² are independently selected from one to four of C₁₋₆alkyl, fluoro-substituted C₁₋₆alkyl, halo, C₁₋₆alkoxy, fluoro-substituted C₁₋₆alkoxy and unsubstituted or substituted phenyl; and/or
adjacent substituents on Q² are joined together to form, including the carbon atoms to which they are attached, one or more unsubstituted or substituted phenylene, naphthylene or ferrocenylene ring systems; or
the term substituted with respect to the Q² substituents means that one or more of the available hydrogen atoms on the group are replaced with C₁₋₆alkyl, fluoro-substituted C₁₋₆alkyl, C₁₋₆alkoxy, fluoro-substituted C₁₋₆alkoxy, halo or C₆₋₁₄aryl; and
Q² is chiral or achiral.

47. The method according to claim 46, wherein R⁸ and R⁹ are both phenyl, tolyl or xylyl.

48. The method according to claim 45, wherein R¹⁰ and R¹¹ and both H or one of R¹⁰ or R¹¹ is joined with a substituent on Q² to form, together with the nitrogen atom to which R¹⁰ and R¹¹ is attached, a substituted or unsubstituted pyridine ring and the other of one of R¹⁰ or R¹¹ is not present.

49. The method according to claim 45, wherein PN is selected from:
Ph₂PCH₂CH₂NH₂(PGly); and 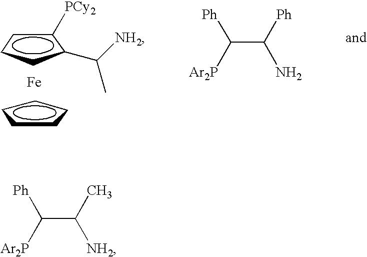
wherein Ar is selected from Ph, tolyl and xylyl; and
optical isomers thereof and mixtures of optical isomers in any ratio.

50. The method according to claim 45, wherein X is defined as in claim 25.

51. The method according to claim 45, wherein LB is defined as in claim 26.

52. The method according to claim 45, wherein Y is defined as in claim 27.

53. The method according to claim 45, wherein the synthetic organic reaction is selected from hydrogenation, transfer hydrogenation, hydroformylation, hydrosilylation, hydroboration, hydroamination, hydrovinylation, hydroarylation, hydration, oxidation, epoxidation, reduction, C—C and C—X bond formation, functional group interconversion, kinetic resolution, dynamic kinetic resolution, cycloaddition, Diels-Alder, retro-Diels-Alder, sigmatropic rearrangement, electrocyclic reactions, ring-opening and/or ring-closing olefin metathesis, carbonylation and aziridination.

54. The method according to claim 53, wherein the C—C and C—X bond formation reaction is selected from Heck, Suzuki-Miyaura, Negishi, Buchwald-Hartwig Amination, α-Ketone Arylation, N-Aryl Amination, Murahashi, Kumada and Stifle reactions.

55. The method according to claim 53, wherein the reaction is hydrogenation or transfer hydrogenation.

56. A process for preparing a compound of formula (II), comprising combining a compound of the formula
Ru(PN)₂X₂ (XII)
wherein PN and X are as defined in claim 45, with one or two molar equivalents of an anion abstracting agent and optionally a non- or weakly-coordinating Lewis Base, and reacting under conditions to form the compound and optionally isolating the compound.

57. A process for preparing a compound of formula (II), comprising combining a precursor ruthenium compound with one or two molar equivalents of an anion abstracting agent, and optionally a Lewis Base and reacting under conditions to form a cationic or dicationic precursor ruthenium compound and combining the cationic or dicationic precursor ruthenium compound with one or more PN ligands as defined in claim 45, and optionally a non- or weakly-coordinating Lewis Base, under conditions to form the compound and optionally isolating the compound.

58. The process according to claim 57, wherein the precursor ruthenium compound is of the formula [RuX₂(p-ligand)]₂ or RuX₂(ligand), wherein X is as defined in claim 50 and ligand is any displaceable ligand.

59. The process according to claim 58, wherein the displaceable ligand is p-cymene, benzene, cyclooctadiene (COD) or norbornadiene (NBD).

60. The process according to claim 59, wherein the displaceable ligand is p-cymene or NBD.

61. The process according to claim 57, wherein the precursor metal compound is of the formula RuX₂(P₂)(LB)ₙ, wherein X is as defined in claim 50, P₂ is as defined in claim 1 and LB is as defined in claim 45 and n is 1 or 2.

62. The process according to claim 61, wherein (P₂) is BINAP and LB is DMF or pyridine.

63. The process according to claim 57, wherein the anion abstracting agent is a salt of a non-coordinating counter anion Y, wherein Y is as defined in claim 52.
